(19) United States
(12) Patent Application Publication
(10) Pub. No.: US 2020/0031769 A1
(43) Pub. Date: Jan. 30, 2020

(54) NOVEL POLYMORPHS OF (5-[3-(3-HYDROXYPHENOXY)AZETIDIN-1-YL]-5-METHYL-2,2-DIPHENYLHEXANAMIDE HYDROCHLORIDE

(71) Applicant: MYLAN LABORATORIES LIMITED, Hyderabad (IN)

(72) Inventors: Ramakoteswara Rao Jetti, Hyderabad (IN); Anjaneyaraju INDUKURI, Hyderabad (IN); Narasimha Murty PILLI, Hyderabad (IN)

(21) Appl. No.: 16/493,492

(22) PCT Filed: Mar. 15, 2018
(86) PCT No.: PCT/IN2018/050148

§ 371 Date: Sep. 12, 2019

(30) Foreign Application Priority Data
Mar. 15, 2017 (IN) 201741008964

(51) Int. Cl. C07D 205/04; A61P 11/00

(52) U.S. Cl. CPC C07D 205/04; C07B 2200/13; A61P 11/00

(57) ABSTRACT

The present disclosure provides solid forms of the hydrochloride salt of (5-[3-(3-Hydroxyphenoxy)azetidin-1-yl]-5-methyl-2,2-diphenylhexanamide (Compound-A). In particular, an amorphous form, an ethyl acetate solvate form, a methyl n-butyl ketone solvate form, an anisole solvate form, an isobutyl acetate solvate form, a n-butyl acetate solvate form, a toluene solvate form, a 4-methyl-2-pentanol solvate form, a n-propyl acetate solvate form, a xylene solvate form, Form VIII and Form IX of the salt are disclosed. Processes for the preparation of each of the disclosed forms are also provided.

### FIELD OF THE INVENTION

The present invention relates generally to active pharmaceutical ingredients and more specifically to the hydrochloride salt of (5-[3-(3-Hydroxyphenoxy)azetidin-1-yl]-5-methyl-2,2-diphenylhexanamide. In particular, an amorphous form, an ethyl acetate solvate form, a methyl n-butyl ketone solvate form, an anisole solvate form, an isobutyl acetate solvate form, a n-butyl acetate solvate form, a toluene solvate form, a 4-methyl-2-pentanol solvate form, a n-propyl acetate solvate form, a xylene solvate form, Form VIII and Form IX of the salt are disclosed. Processes for the preparation of each of the disclosed forms are also provided.

### BACKGROUND OF THE INVENTION

(5-[3-(3-Hydroxyphenoxy)azetidin-1-yl]-5-methyl-2,2-diphenylhexanamide hydrochloride has a structure depicted below as Compound-A.

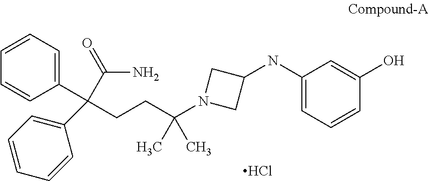 [SMILES: CC(C)(CCC(C(N)=O)(c1ccccc1)c1ccccc1)N1CC(Nc2cccc(O)c2)C1 ; Cl]

Compound-A is a muscarinic antagonist useful for treating allergy or respiratory chronic obstructive pulmonary disease.

Compound-A and pharmaceutically acceptable salts are claimed in U.S. Pat. No. 7,772,223 B2 and one of its non-solvated crystalline forms is claimed in U.S. Pat. No. 8,263,583 B2.

The present disclosure provides a wide range of polymorphic forms of Compound-A such as an amorphous form, an ethyl acetate solvate form, a methyl n-butyl ketone solvate form, an anisole solvate form, an isobutyl acetate solvate form, a n-butyl acetate solvate form, a toluene solvate form, a 4-methyl-2-pentanol solvate form, a n-propyl acetate solvate form, a xylene solvate form, Form VIII and Form IX. Processes for the preparation of each of the disclosed forms are also provided.

### SUMMARY OF THE INVENTION

In one aspect, the present invention provides an amorphous form of Compound-A. In another aspect, the present invention provides a process for the preparation of the amorphous form of Compound-A. In one embodiment, the amorphous form of Compound-A can be prepared by a process that includes the following steps:
a) dissolving Compound-A in a solvent; and
b) removing the solvent to isolate amorphous form of Compound-A.

In another aspect, the present invention provides crystalline Form I of ethyl acetate solvate of Compound-A and a process for the preparation of the same. In one embodiment, crystalline Form I of ethyl acetate solvate of Compound-A can be prepared by a process that includes the following steps:
a) suspending Compound-A in ethyl acetate,
b) adding a second solvent,
c) cooling the reaction mixture, and
d) isolating crystalline Form I of ethyl acetate solvate of Compound-A.

In another embodiment, crystalline Form I of ethyl acetate solvate of Compound-A can be prepared by a process that includes the following steps:
a) dissolving Compound-A in a solvent,
b) adding ethyl acetate,
c) optionally seeding with crystalline form I of Compound-A, and
d) isolating crystalline Form I of ethyl acetate solvate of Compound-A.

In another aspect, the present invention provides crystalline Form II of methyl n-butyl ketone solvate of Compound-A and a process for the preparation of the same. In one embodiment, crystalline Form II of methyl n-butyl ketone solvate of Compound-A can be prepared by a process that includes the following steps:
a) suspending Compound-A in methyl n-butyl ketone,
b) optionally cooling the reaction mixture, and
c) isolating crystalline Form II of methyl n-butyl ketone solvate of Compound-A.

In yet another embodiment, crystalline Form II of methyl n-butyl ketone solvate of Compound-A can be prepared by a process that includes the following steps:
a) suspending Compound-A in methyl n-butyl ketone,
b) adding a second solvent, and
c) isolating crystalline Form II of methyl n-butyl ketone solvate of Compound-A.

In another aspect, the present invention provides crystalline Form III of anisole solvate of Compound-A and a process for the preparation of the same. In one embodiment, crystalline Form III of anisole solvate of Compound-A can be prepared by a process that includes the following steps:
a) dissolving Compound-A in suitable solvent,
b) adding anisole, and
c) isolating crystalline Form III of anisole solvate of Compound-A.

In another embodiment, crystalline Form III of anisole solvate of Compound-A can be prepared by a process that includes the following steps:
a) suspending Compound-A in anisole, and
b) isolating crystalline Form III of anisole solvate of Compound-A.

In another aspect, the present invention provides crystalline Form IV of isobutyl acetate solvate of Compound-A and a process for the preparation of the same. In one embodiment, crystalline Form IV of isobutyl acetate solvate of Compound-A can be prepared by a process that includes the following steps:
a) dissolving Compound-A in suitable solvent,
b) adding isobutyl acetate, and
c) isolating crystalline Form IV of isobutyl acetate solvate of Compound-A.

In another embodiment, crystalline Form IV of isobutyl acetate solvate of Compound-A can be prepared by a process that includes the following steps:
a) suspending Compound-A in isobutyl acetate, and
b) isolating crystalline Form IV of isobutyl acetate solvate of Compound-A.

In another aspect, the present invention provides crystalline Form V of n-butyl acetate solvate of Compound-A and a process for the preparation of the same. In one embodiment, crystalline Form V of n-butyl acetate solvate of Compound-A can be prepared by a process that includes the following steps:
a) dissolving Compound-A in suitable solvent,
b) adding n-butyl acetate, and
c) isolating crystalline Form V of n-butyl acetate solvate of Compound-A.

In another embodiment, crystalline Form V of n-butyl acetate solvate of Compound-A can be prepared by a process that includes the following steps:
a) suspending Compound-A in n-butyl acetate, and
b) isolating Form V of n-butyl acetate solvate of Compound-A.

In another aspect, the present invention provides crystalline Form VI of toluene solvate of Compound-A and a process for the preparation of the same. In one embodiment, crystalline Form VI of toluene solvate of Compound-A can be prepared by a process that includes the following steps:
a) suspending Compound-A in toluene, and
b) isolating crystalline Form VI of toluene solvate of Compound-A.

In another aspect, the present invention provides crystalline Form VII of 4-methyl 2-pentanol solvate of Compound-A and a process for the preparation of the same. In one embodiment, crystalline Form VII of 4-methyl 2-pentanol solvate of Compound-A can be prepared by a process that includes the following steps:
a) suspending Compound-A in 4-methyl 2-pentanol, and
b) isolating crystalline Form VII of 4-methyl 2-pentanol solvate of Compound-A.

In another aspect, the present invention provides crystalline Form VIII of Compound-A and a process for the preparation of the same. In one embodiment, crystalline Form VIII of Compound-A can be prepared by a process that includes the following steps:
a) drying form I of Compound-A, and
b) isolating crystalline Form VIII of Compound-A.

In another aspect, the present invention provides crystalline Form IX of Compound-A and a process for the preparation of the same. In one embodiment, crystalline Form IX of Compound-A can be prepared by a process that includes the following steps:
a) drying form II of Compound-A, and
b) isolating crystalline Form IX of Compound-A.

In another aspect, the present invention provides crystalline Form X of n-propyl acetate solvate of Compound-A and a process for the preparation of the same. In one embodiment, crystalline Form X of n-propyl acetate solvate of Compound-A can be prepared by a process that includes the following steps:
a) suspending Compound-A in n-propyl acetate, and
b) isolating crystalline Form X of n-propyl acetate solvate of Compound-A.

In another aspect, the present invention provides crystalline Form XI of xylene solvate of Compound-A and a process for the preparation of the same. In one embodiment, crystalline Form XI of xylene solvate of Compound-A can be prepared by a process that includes the following steps:
a) suspending Compound-A in xylene, and
b) isolating crystalline Form XI of xylene solvate of Compound-A.

### BRIEF DESCRIPTION OF THE DRAWINGS

Further aspects of the present disclosure together with additional features contributing thereto and advantages accruing therefrom will be apparent from the following description of embodiments of the disclosure which are shown in the accompanying figures wherein:

FIG. 1: PXRD pattern of amorphous Compound-A;

FIG. 2: MDSC thermogram of amorphous Compound-A;

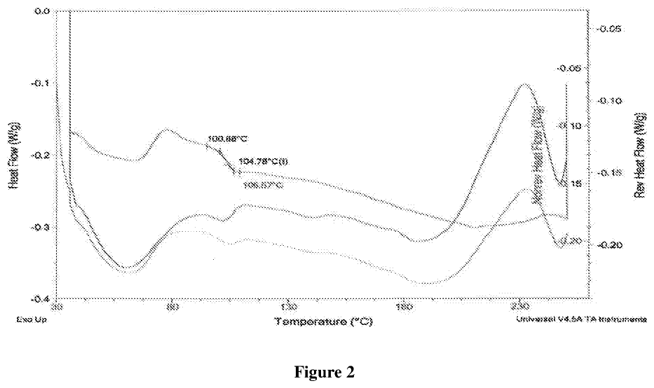

FIG. 3: TGA thermogram of amorphous Compound-A;

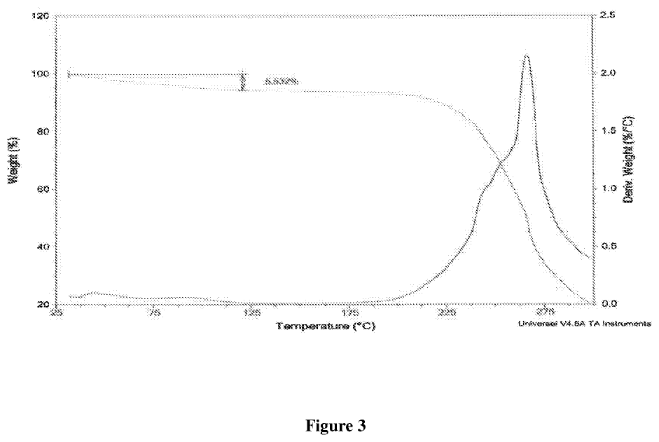

FIG. 4: ¹H NMR spectrum of amorphous Compound-A;

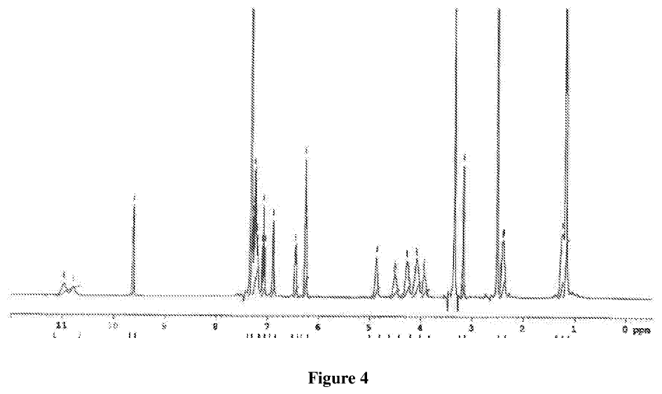

FIG. 5: PXRD pattern of crystalline Form I—ethyl acetate solvate of Compound-A;

FIG. 6: DSC thermogram of crystalline Form I—ethyl acetate solvate of Compound-A;

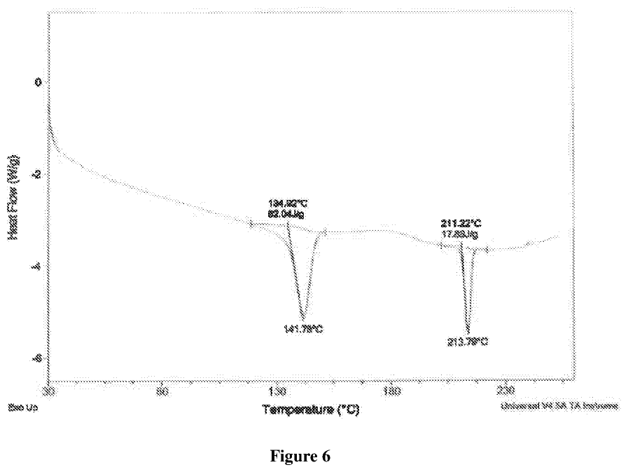

FIG. 7: TGA thermogram of crystalline Form I—ethyl acetate solvate of Compound-A;

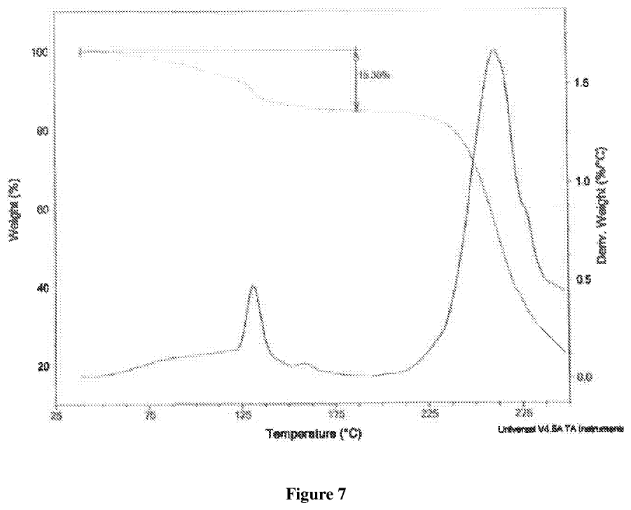

FIG. 8: ¹H NMR spectrum of crystalline Form I—ethyl acetate solvate of Compound-A;

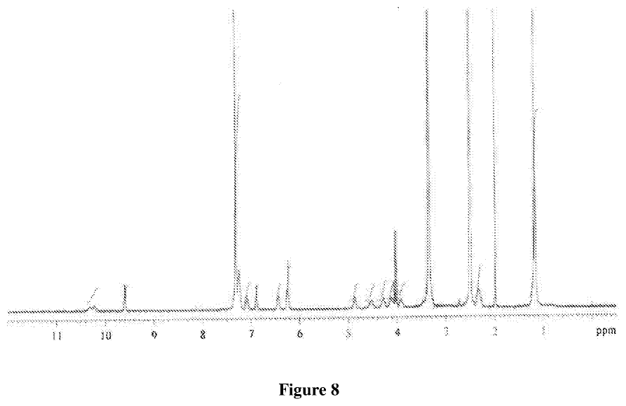

FIG. 9: PXRD pattern of crystalline Form II—methyl n-butyl ketone solvate of Compound-A;

FIG. 10: DSC thermogram of crystalline Form II—methyl n-butyl ketone solvate of Compound-A;

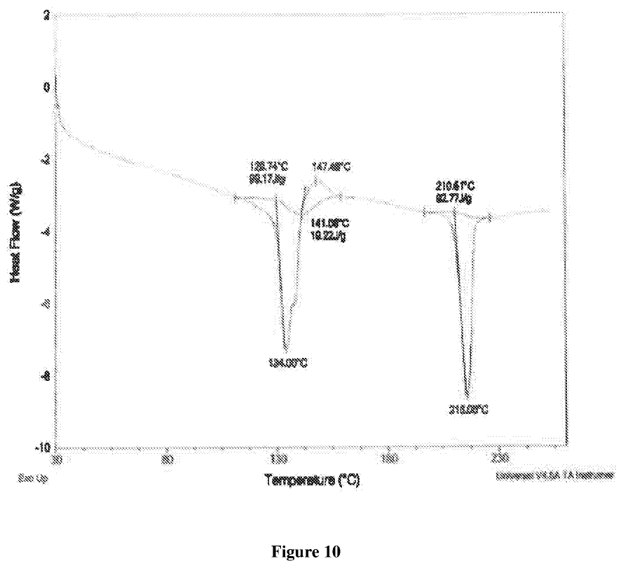

FIG. 11: TGA thermogram of crystalline Form II—methyl n-butyl ketone solvate of Compound-A;

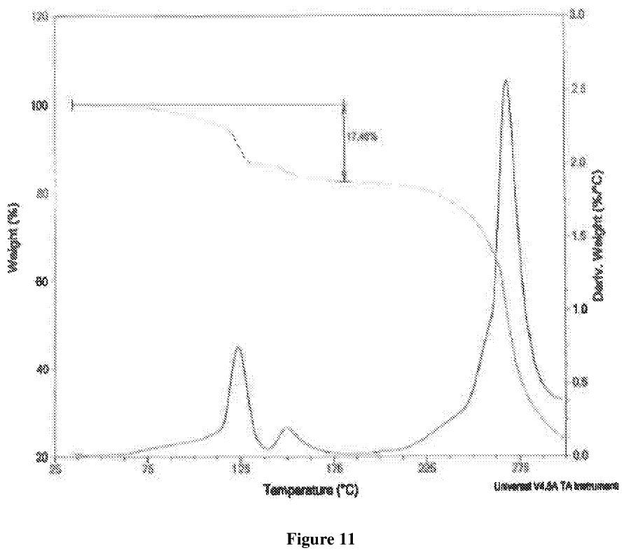

FIG. 12: ¹H NMR spectrum of crystalline Form II—methyl n-butyl ketone solvate of Compound-A;

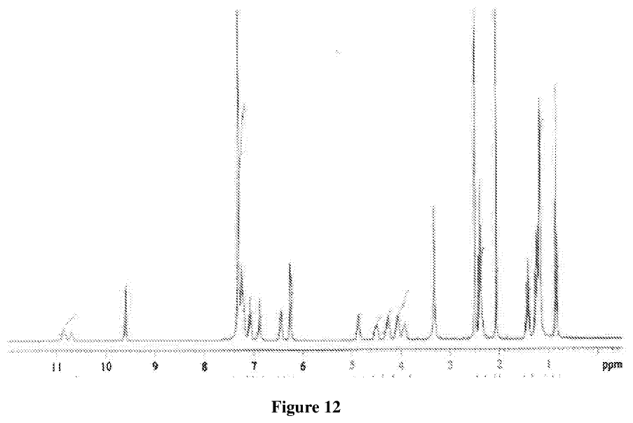

FIG. 13: PXRD pattern of crystalline Form III—anisole solvate of Compound-A;

FIG. 14: DSC thermogram of crystalline Form III—anisole solvate of Compound-A;

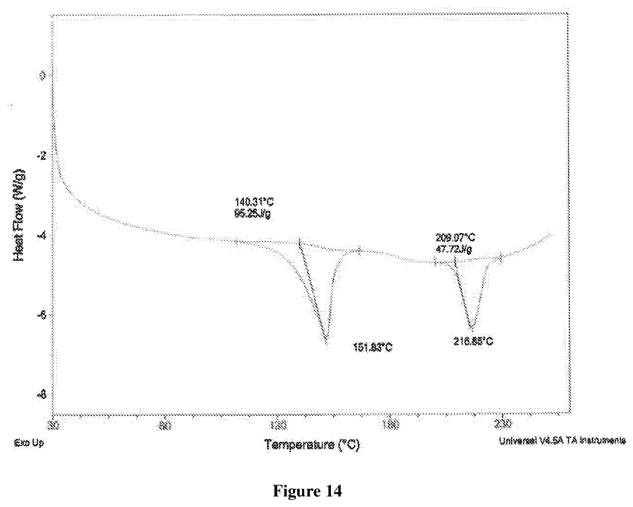

FIG. 15: TGA thermogram of crystalline Form III—anisole solvate of Compound-A;

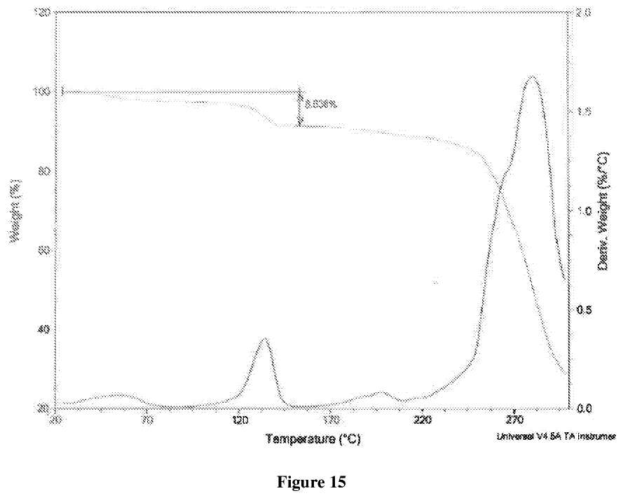

FIG. 16: ¹H NMR spectrum of crystalline Form III—anisole solvate of Compound-A;

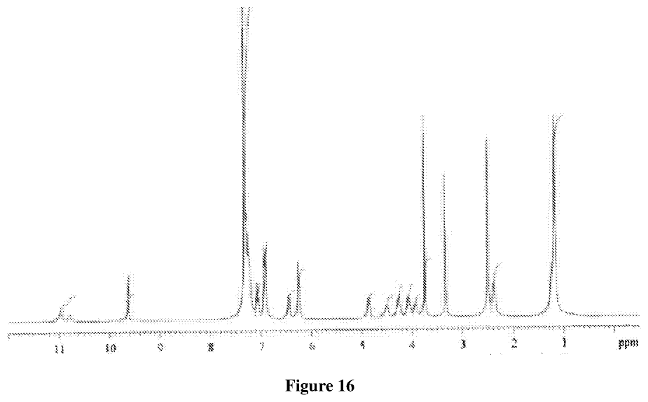

FIG. 17: PXRD pattern of crystalline Form IV—isobutyl acetate solvate of Compound-A;

FIG. 18: DSC thermogram of crystalline Form IV—isobutyl acetate solvate of Compound-A;

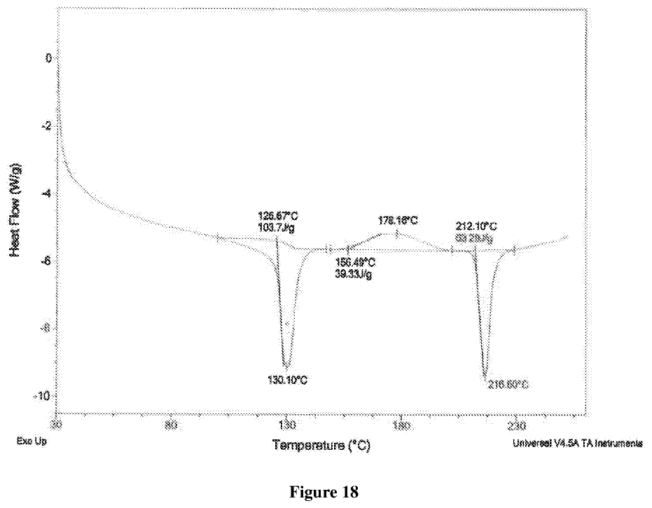

FIG. 19: TGA thermogram of crystalline Form IV—isobutyl acetate solvate of Compound-A;

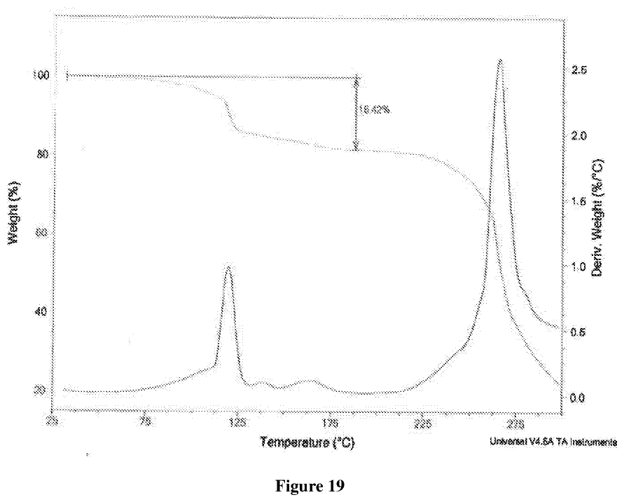

FIG. 20: ¹H NMR spectrum of crystalline Form IV—isobutyl acetate solvate of Compound-A;

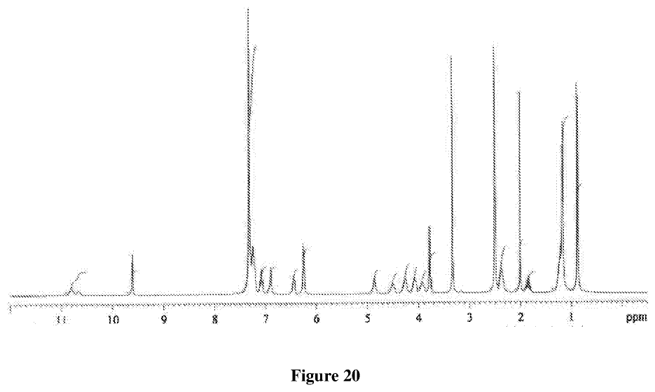

FIG. 21: PXRD pattern of crystalline Form V—n-butyl acetate solvate of Compound-A;

FIG. 22: DSC thermogram of crystalline Form V—n-butyl acetate solvate of Compound-A;

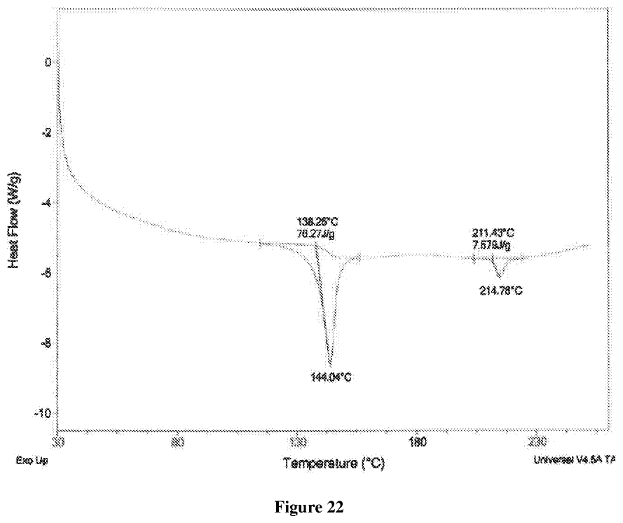

FIG. 23: TGA thermogram of crystalline Form V—n-butyl acetate solvate of Compound-A;

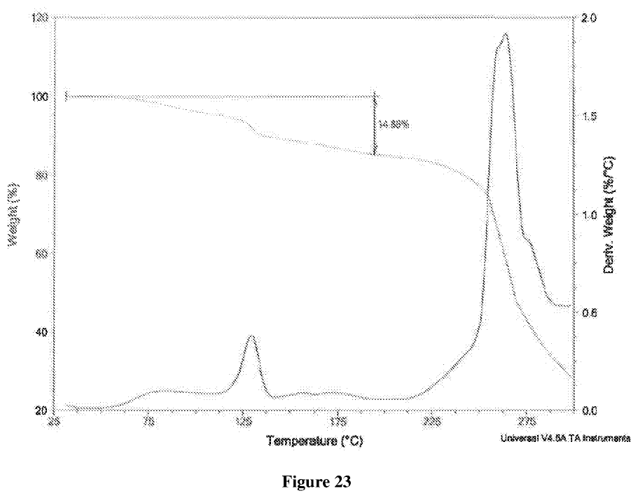

FIG. 24: ¹H NMR spectrum of crystalline Form V—n-butyl acetate solvate of Compound-A;

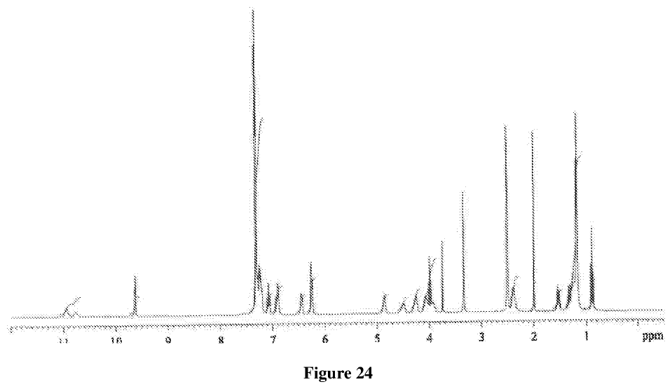

FIG. 25: PXRD pattern of crystalline Form VI—toluene solvate of Compound-A;

FIG. 26: DSC thermogram of crystalline Form VI—toluene solvate of Compound-A;

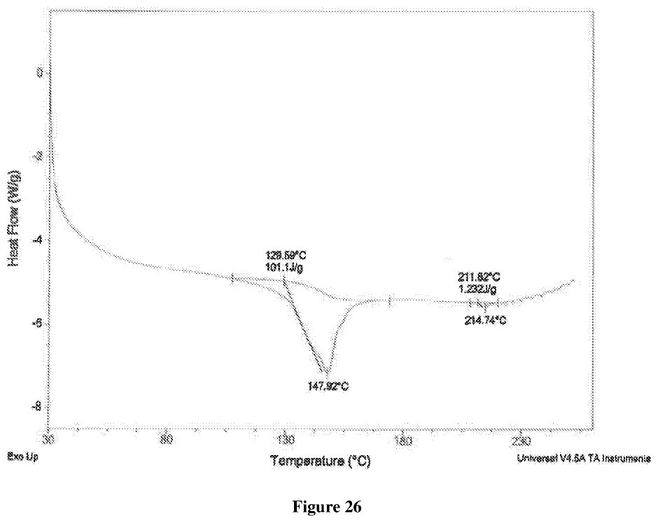

FIG. 27: TGA thermogram of crystalline Form VI—toluene solvate of Compound-A;

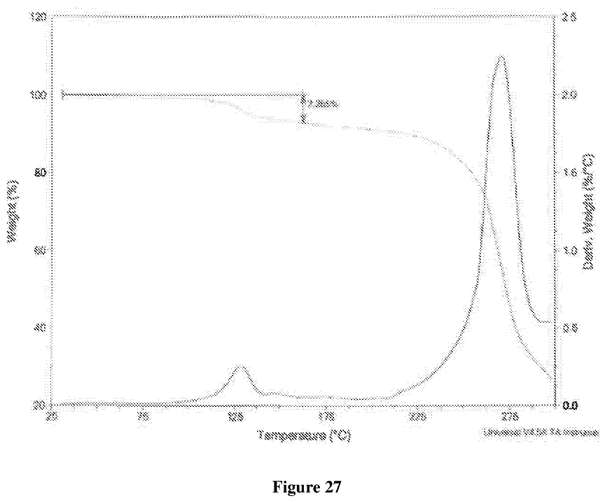

FIG. 28: ¹H NMR spectrum of crystalline Form VI—toluene solvate of Compound-A;

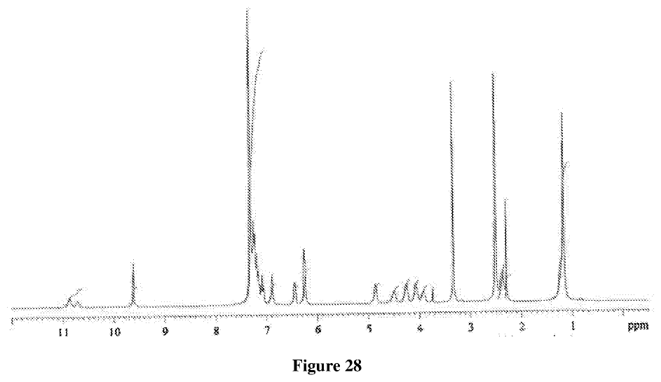

FIG. 29: PXRD pattern of crystalline Form VII—4-methyl-2-pentanol solvate of Compound-A;

FIG. 30: DSC thermogram of crystalline Form VII—4-methyl-2-pentanol solvate of Compound-A;

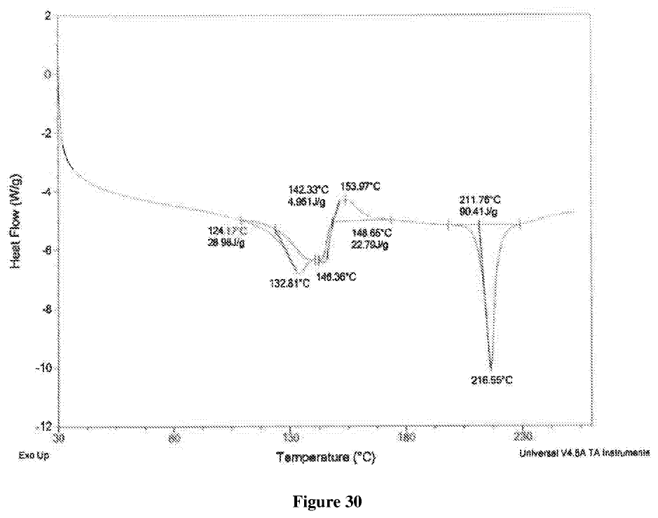

FIG. 31: TGA thermogram of crystalline Form VII—4-methyl-2-pentanol solvate of Compound-A;

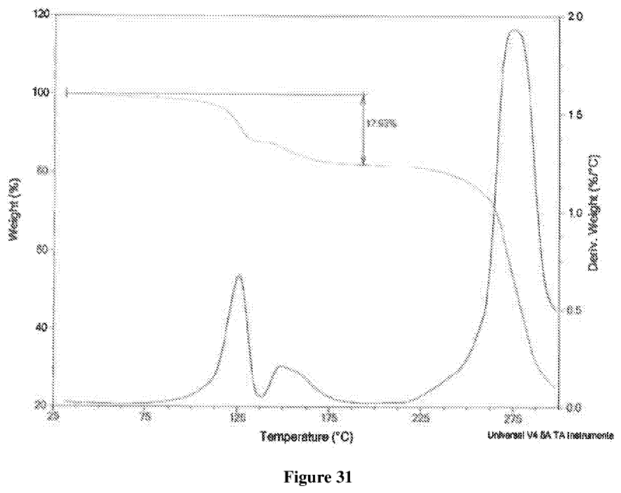

FIG. 32: ¹H NMR spectrum of crystalline Form VII—4-methyl-2-pentanol solvate of Compound-A;

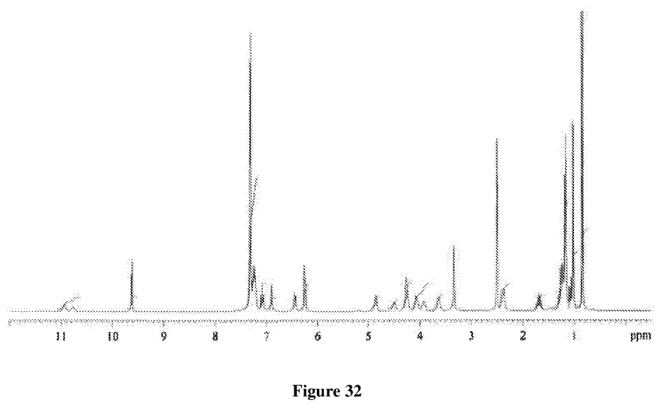

FIG. 33: PXRD pattern of crystalline Form VIII of Compound-A;

FIG. 34: DSC thermogram of crystalline Form VIII of Compound-A;

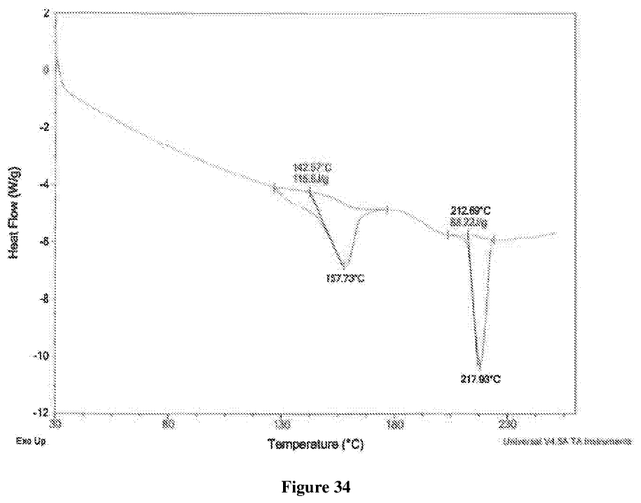

FIG. 35: TGA thermogram of crystalline Form VIII of Compound-A;

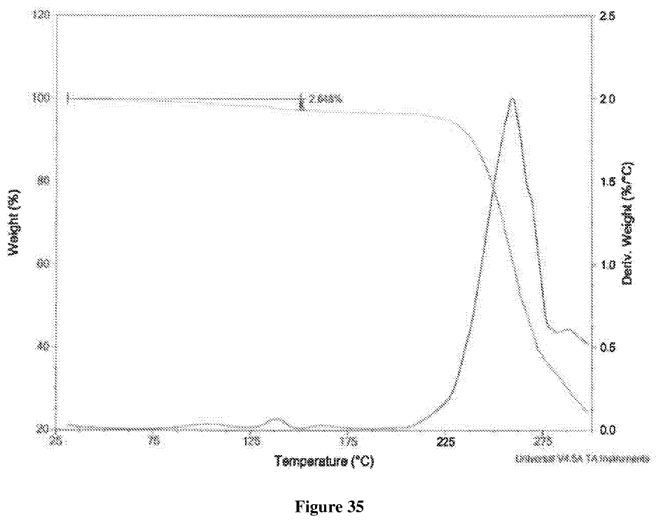

FIG. 36: PXRD pattern of crystalline Form IX of Compound-A;

FIG. 37: DSC thermogram of crystalline Form IX of Compound-A;

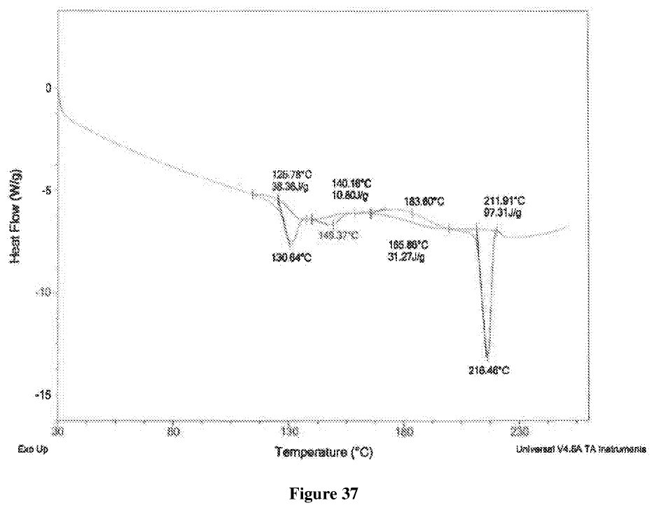

FIG. 38: TGA thermogram of crystalline Form IX of Compound-A;

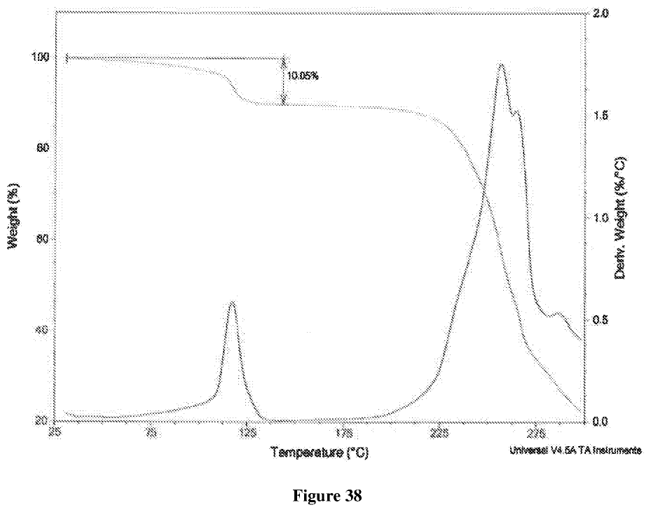

FIG. 39: PXRD pattern of crystalline Form X—n-propyl acetate solvate of Compound-A;

FIG. 40: DSC thermogram of crystalline Form X—n-propyl acetate solvate of Compound-A;

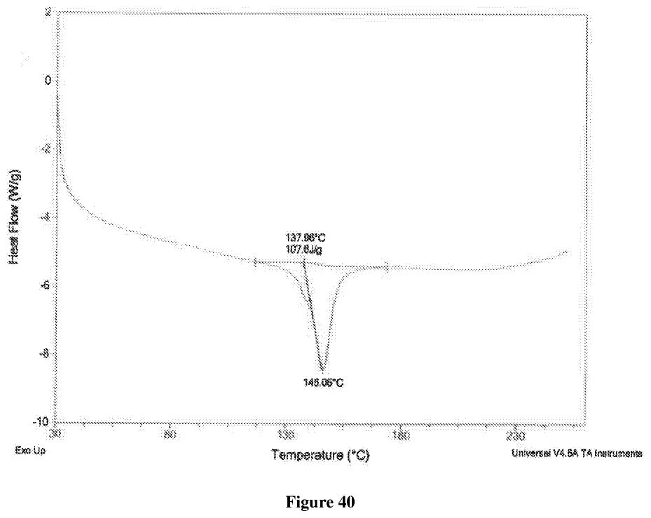

FIG. 41: TGA thermogram of crystalline Form X—n-propyl acetate solvate of Compound-A;

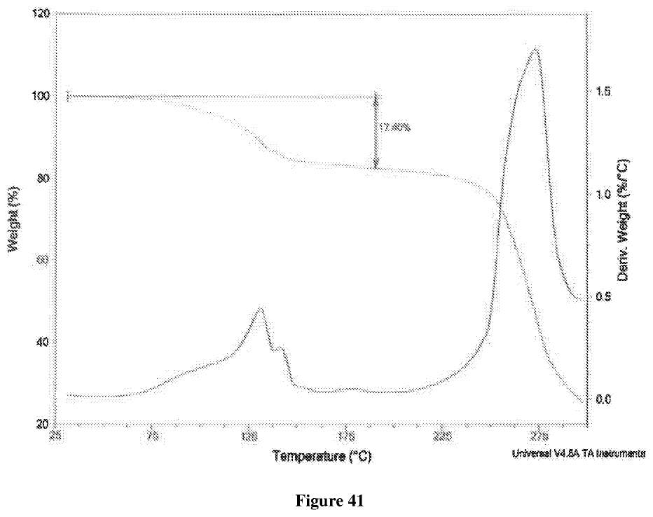

FIG. 42: ¹H NMR spectrum of crystalline Form X—n-propyl acetate solvate of Compound-A;

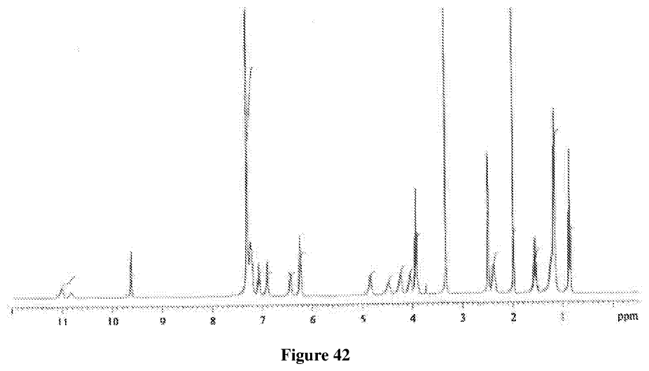

FIG. 43: PXRD pattern of crystalline Form XI—xylene solvate of Compound-A;

FIG. 44: DSC thermogram of crystalline Form XI—xylene solvate of Compound-A;

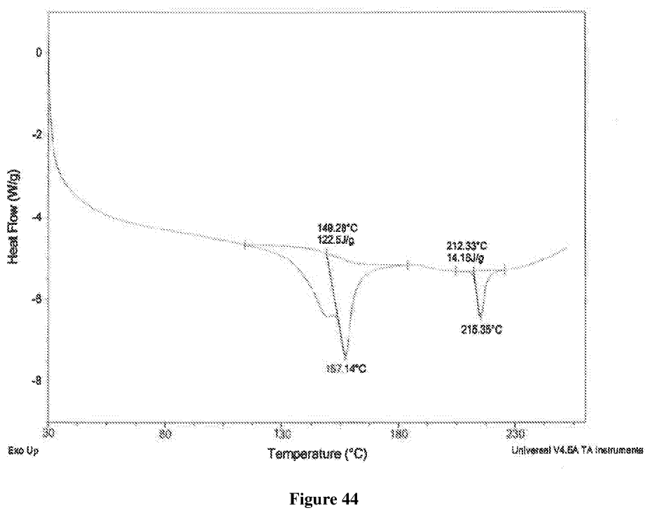

FIG. 45: TGA thermogram of crystalline Form XI—xylene solvate of Compound-A; and

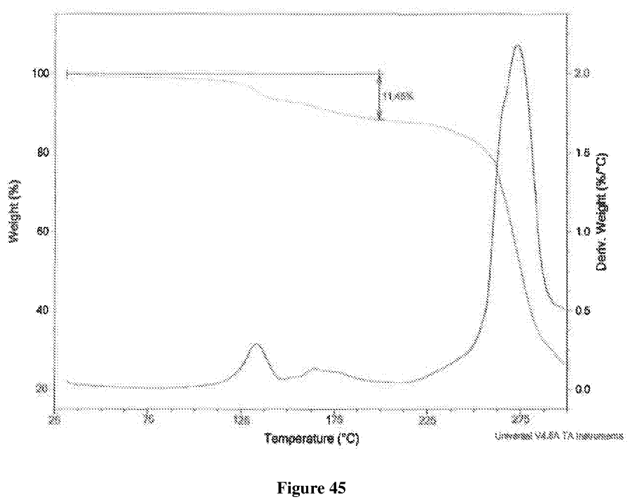

FIG. 46: ¹H NMR spectrum of crystalline Form XI—xylene solvate of Compound-A.

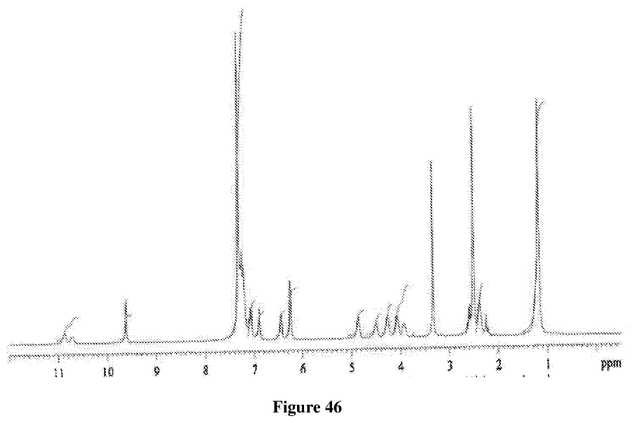

### DETAILED DESCRIPTION OF THE INVENTION

In one aspect, the present invention provides an amorphous form of Compound-A. In another aspect, the present invention provides solvates of Compound-A. In still another aspect, the present invention provides processes for making the various novel forms of Compound-A disclosed herein.

### Instrumentation Details:

The PXRD measurements were carried out using a BRUKER D8 Discover powder diffractometer equipped with goniometer of θ/2θ configuration and a Lynx Eye detector. The Cu-anode X-ray tube was operated at 40 kV and 40 mA. The experiments were conducted over the 2θ range of 2.0°-50.0°, 0.030° step size and 0.2 seconds step time.

### Differential Scanning Calorimetry (DSC)

Differential Scanning calorimetry of novel forms were measured on TA Q1000 of TA instruments. The experiment was conducted from 30° C. to 250° C. at a heating rate of 20.0° C./min and nitrogen purging at a flow rate of 50 ml/min Standard aluminum pans covered by lids with pin holes were used.

Differential Scanning calorimetry of an amorphous form was measured on TA Q1000 of TA instruments. The samples were heated from 30° C. to 250° C. at a heating rate of 5.0° C./min with modulation amplitude±1.0° C., modulation period 60 seconds and nitrogen purging at a flow rate of 50 ml/min Standard aluminum pans covered by lids with five pin holes were used. The glass transition temperature (Tg) of the amorphous form was measured using modulated DSC software.

### Thermo Gravimetric Analysis (TGA)

TGA was recorded using on instrument TA Q5000 of TA instruments. The experiments were performed at a heating rate of 10.0° C./min over a temperature range of 25° C.−300° C. purging with nitrogen at a flow rate of 25 ml/min.

### Nuclear Magnetic Resonance (NMR) Spectroscopy

The ¹HNMR experiments were performed on a Bruker 300 MHz Avance NMR spectrometer equipped with a 5 mm BBO probe in DMSO-d6. The data was collected and processed by Top Spin-NMR software.

In one aspect, the present invention provides an amorphous form of Compound-A.

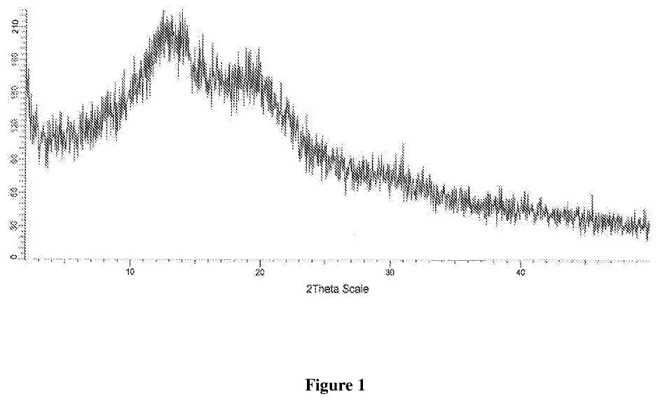

Within the context of this embodiment, amorphous Compound-A is prepared by the methods disclosed herein and may be characterized as amorphous by the PXRD pattern in FIG. 1.

In another aspect, the present invention provides a process for the preparation of the amorphous form of Compound-A.

In one embodiment, the amorphous form of Compound-A can be prepared by a process that includes the following steps:
a) dissolving Compound-A in a solvent; and

b) removing the solvent to isolate amorphous form of Compound-A.

Within the context of this embodiment, Compound-A is dissolved in a suitable solvent, for example polar solvents; selected from alcoholic solvent; such as methanol, ethanol, isopropanol, 1-propanol, n-butanol, 2-butanol, isobutanol, t-butanol, 2-methoxy ethanol, 2-ethoxy ethanol, and mixtures thereof.

Next, the solvent may be removed from the solution to isolate an amorphous form of Compound-A. Solvent removal may be carried out by techniques well known in the art, such as evaporation, distillation, spray drying, lyophilization, agitated thin film drying, or combinations thereof. In certain embodiments of the present disclosure, the technique of spray drying is particularly useful for removing the solvent.

In one aspect, the present invention provides crystalline Form I of ethyl acetate solvate of Compound-A.

Within the context of the present invention, crystalline Form I of ethyl acetate solvate of Compound-A prepared by methods disclosed herein may be characterized by PXRD spectrum having peaks (±0.2° 2θ) 6.83, 9.58, 10.91, 14.29, 19.71, 20.04, 21.59, 22.14, and 27.65.

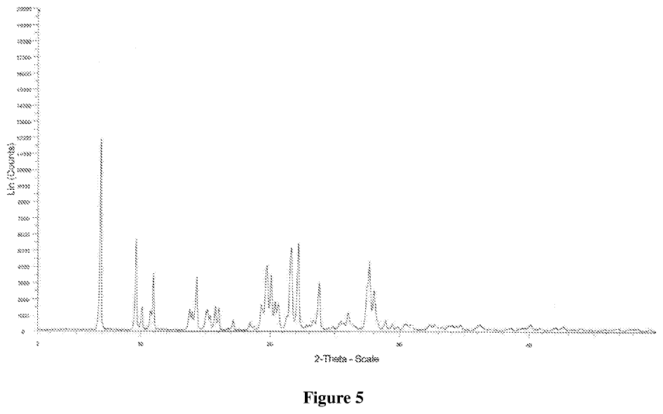

The crystalline Form I of ethyl acetate solvate of Compound-A prepared by methods disclosed herein may be characterized by the PXRD pattern in FIG. 5.

In one embodiment, crystalline Form I of ethyl acetate solvate of Compound-A can be prepared by a process that includes the following steps:
a) suspending Compound-A in ethyl acetate,
b) adding a second solvent,
c) cooling the reaction mixture, and
d) isolating crystalline Form I of ethyl acetate solvate of Compound-A.

Within the context of this embodiment, Compound-A is suspended in ethyl acetate and added a suitable second solvent. In some embodiments, depending on the solvent used, it is useful to dissolve Compound-A in the solvent at an elevated temperature. One of skill in the art will be able to determine the appropriate solvent and temperature conditions needed to dissolve Compound-A in a solvent without undue experimentation. For example, in some particularly useful embodiments, Compound-A is suspended in ethyl acetate at about 55° C. to about 65° C.

The suitable second solvent for addition is a polar solvent. Examples of polar solvents include, but are not limited to, formic acid, acetic acid, methanol, ethanol, isopropanol, 1-propanol, n-butanol, 2-butanol, isobutanol, t-butanol, 2-methoxy ethanol, 2-ethoxy ethanol, dimethyl sulfoxide, N,N dimethyl formamide, N,N dimethyl acetamide, N-Methyl-2-pyrrolidone and mixtures thereof. In some particularly useful embodiments, formic acid is used as second solvent.

The above resulted reaction mixture is cooled to 20-35° C.; In some particularly useful embodiments, the solvent may be removed from the solution at 25-30° C. to isolate crystalline form I of ethyl acetate solvate of Compound-A. Solvent removal may be carried out by techniques well known in the art, such as evaporation and distillation. In certain embodiments of the present disclosure, slow evaporation of solvent is useful for removing the solvent.

Next, Form I of ethyl acetate solvate of Compound-A may be isolated. For example, the solid obtained above was filtered to yield crystalline Form I of ethyl acetate solvate of Compound-A.

In another embodiment, crystalline Form I of ethyl acetate solvate of Compound-A can be prepared by a process that includes the following steps:
a) dissolving Compound-A in a solvent,
b) adding ethyl acetate,
c) optionally seeding with crystalline form I of Compound-A, and
d) isolating crystalline Form I of ethyl acetate solvate of Compound-A.

Within the context of this embodiment, Compound-A is dissolved in suitable solvent at 65-80° C. In some particularly useful embodiments of the present disclosure, Compound-A is dissolved at about 70° C. to about 75° C. For example the suitable solvent is a polar solvent. Examples of polar solvents include, but are not limited to, formic acid, acetic acid, methanol, ethanol, isopropanol, 1-propanol, n-butanol, 2-butanol, isobutanol, t-butanol, 2-methoxy ethanol, 2-ethoxy ethanol, dimethyl sulfoxide, N,N dimethyl formamide, N,N dimethyl acetamide, N-Methyl-2-pyrrolidone and mixtures thereof. In some particularly useful embodiments, formic acid is used as a solvent.

The obtained reaction mixture is cooled to 20-35° C. (in some particularly useful embodiment's 25-30° C.) and added ethyl acetate solvent. Further, the reaction mixture is optionally seeded with Form I. In some particular embodiment of the present invention, the reaction mixture is seeded with crystalline Form I.

In some embodiments, stirring or agitation may be carried out at a temperature of about 15° C. to about 40° C. In some embodiments, a temperature of about 25° C. to about 30° C. is used. In some embodiments, the stirring or agitation may be carried out for about 2 hours to about 5 days. In some particularly useful embodiments of the present disclosure, stirring the solution is carried out for 3 days.

Next, Form I of ethyl acetate solvate of Compound-A may be isolated. This may be carried out by methods well-known in the art. For example, the suspension may be filtered to isolate solid crystalline Form I of ethyl acetate solvate of Compound-A.

In one aspect, the present invention provides crystalline Form II of methyl n-butyl ketone solvate of Compound-A.

Within the context of the present invention, crystalline Form II of methyl n-butyl ketone solvate of Compound-A prepared by methods disclosed herein may be characterized by PXRD spectrum having peaks (±0.2° 2θ) 6.61, 20.80, and 26.81.

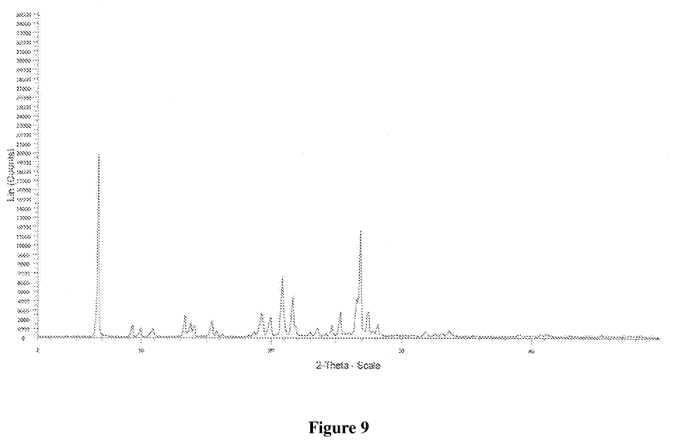

The crystalline Form II of methyl n-butyl ketone solvate of Compound-A prepared by methods disclosed herein may be characterized by the PXRD pattern in FIG. 9.

In another embodiment, crystalline Form II of methyl n-butyl ketone solvate of Compound-A can be prepared by a process that includes the following steps:
a) suspending Compound-A in methyl n-butyl ketone,
b) optionally cooling the reaction mixture, and
c) isolating crystalline Form II of methyl n-butyl ketone solvate of Compound-A.

Within the context of this embodiment, Compound-A is suspended in methyl n-butyl ketone. In some embodiments suspending Compound-A is carried at 20-30° C., In some particularly useful embodiment of the present disclosure at 20-25° C. In another embodiment, suspending Compound-A in methyl n-butyl ketone is carried out at 65-80° C. In some particularly useful embodiments, at 70-75° C. Further, the suspension is optionally cooled to −20 to 30° C., in some particularly useful embodiments, 20-25° C. In some particularly useful embodiments, −20° C.

In some embodiments it is useful to maintain the reaction mixture at the same temperature for about 3-5 days. In some particularly useful embodiments of the present disclosure, the temperature is maintained for about 2 days after which Form II of methyl n-butyl ketone solvate of Compound-A may be isolated. For example, the suspension may be filtered to obtain solid crystalline Form II of methyl n-butyl ketone solvate of Compound-A.

Yet another embodiment, crystalline Form II of methyl n-butyl ketone solvate of Compound-A can be prepared by a process that includes the following steps:
a) suspending Compound-A in methyl n-butyl ketone,
b) adding a second solvent, and
c) isolating crystalline Form II of methyl n-butyl ketone solvate of Compound-A.

Within the context of this embodiment, Compound-A is suspended in methyl n-butyl ketone. In some embodiments suspending Compound-A is carried out at 20-30° C. In some particularly useful embodiments of the present disclosure it is carried out at 20-25° C. In another embodiment, suspending Compound-A in methyl n-butyl ketone is carried out at 65-80° C. In some particularly useful embodiments it is carried out at 70-75° C. The second solvent is added to the suspension. For example the suitable second solvent is a polar solvent. Examples of polar solvents include, but are not limited to, methanol, ethanol, isopropanol, 1-propanol, n-butanol, 2-butanol, isobutanol, t-butanol, 2-methoxy ethanol, 2-ethoxy ethanol, formic acid and acetic acid, dimethyl sulfoxide, N,N dimethyl formamide, N,N dimethyl acetamide, N-Methyl-2-pyrrolidone and mixtures thereof. In some particularly useful embodiments, methanol is used as a solvent.

Within the context of the above embodiment, the reaction mixture is cooled to −20 to 30° C., in some useful embodiments to 20-25° C. and, in some particularly useful embodiments, to −20° C. In some embodiments it is useful to maintain the reaction mixture at the same temperature for about 3-5 days. In some particularly useful embodiments the temperature is maintained for about 2 days.

Next, Form II of methyl n-butyl ketone solvate of Compound-A may be isolated. For example, the suspension may be filtered to isolate solid crystalline Form II of methyl n-butyl ketone solvate of Compound-A.

In one aspect, the present invention provides crystalline Form III of anisole solvate of Compound-A.

Within the context of the present invention, crystalline Form III of anisole solvate of Compound-A prepared by methods disclosed herein may be characterized by PXRD spectrum having peaks (±0.2° 2θ) 6.86, 19.7 and 22.36.

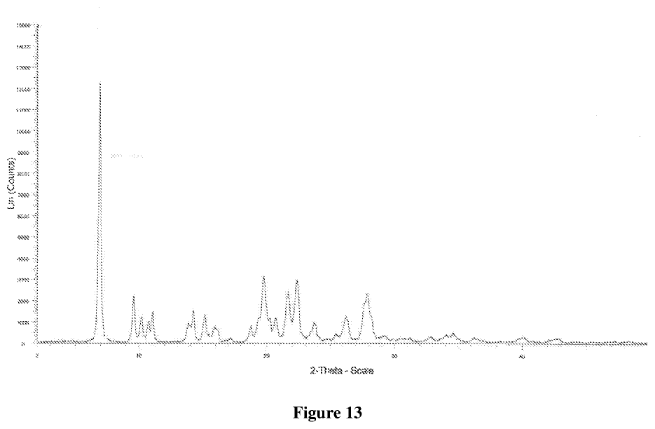

The crystalline Form III of anisole solvate of Compound-A prepared by methods disclosed herein may be characterized by the PXRD pattern in FIG. 13.

In another embodiment, crystalline Form III of anisole solvate of Compound-A can be prepared by a process that includes the following steps:
a) dissolving Compound-A in suitable solvent,
b) adding anisole, and
c) isolating crystalline Form III of anisole solvate of Compound-A.

Within the context of this embodiment, Compound-A is dissolved in suitable solvent at 65-80° C., in some particularly useful embodiments at 70-75° C. Preferably, the suitable second solvent is a polar solvent. Examples of polar solvents include, but are not limited to, formic acid, acetic acid, methanol, ethanol, isopropanol, 1-propanol, n-butanol, 2-butanol, isobutanol, t-butanol, 2-methoxy ethanol, 2-ethoxy ethanol, dimethyl sulfoxide, N,N dimethyl formamide, N,N dimethyl acetamide, N-Methyl-2-pyrrolidone and mixtures thereof. In some particularly useful embodiments, formic acid is used as a solvent.

Within the context of the above embodiment, the obtained reaction mixture is cooled to 20-35° C., in some particularly useful embodiments at 25-30° C. The solvent may be removed from the solution to isolate crystalline Form III of anisole solvate of Compound-A. Solvent removal may be carried out by techniques well known in the art, such as evaporation, distillation, vacuum drying or combinations thereof. In certain embodiments of the present disclosure, slow evaporation is useful for removing the solvent.

Next, Form III of methyl n-butyl ketone solvate of Compound-A may be isolated. For example, the suspension may be filtered to isolate solid crystalline Form III of anisole solvate of Compound-A.

In another embodiment, crystalline Form III of anisole solvate of Compound-A can be prepared by a process that includes the following steps:
a) suspending Compound-A in anisole, and
b) isolating crystalline Form III of anisole solvate of Compound-A.

Within the context of this embodiment, Compound-A is suspended in anisole. In some embodiments, it is found useful to maintain the reaction for about 2 hours to about 5 days. In some particularly useful embodiments it is found useful to maintain the reaction for about 1-3 days. In certain embodiments of the present disclosure, it is useful to maintain the reaction for 16 to 24 hours.

Next, the solvent is removed from the suspension of Compound-A. Solvent removal may be carried out by techniques well known in the art, such as evaporation, distillation, vacuum drying or combinations thereof. In certain embodiments of the present disclosure vacuum drying of solvent is useful for removing the solvent to isolate crystalline Form III of anisole solvate of Compound-A.

In some embodiment of the present disclosure, the solid obtained after removing the solvent may be filtered and further subjected to drying to yield crystalline Form III of anisole solvate of Compound-A.

In one aspect, the present invention provides crystalline Form IV of isobutyl acetate solvate of Compound-A.

Within the context of the present invention, crystalline Form IV of isobutyl acetate solvate of Compound-A prepared by methods disclosed herein may be characterized by PXRD spectrum and having peaks (±0.2° 2θ) 6.68, 8.92, 11.01, 13.32, 13.83, 15.80, 18.60, 19.26, 19.56, 20.11, 20.37, 22.10, 23.76, 25.16, 25.96, 26.94, and 28.00.

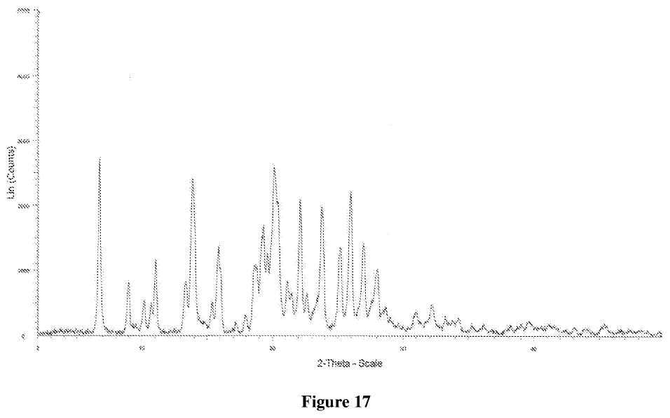

The crystalline Form IV of isobutyl acetate solvate of Compound-A prepared by methods disclosed herein may be characterized by the PXRD pattern in FIG. 17.

In another embodiment, crystalline Form IV of isobutyl acetate solvate of Compound-A can be prepared by a process that includes the following steps:
a) dissolving Compound-A in suitable solvent,
b) adding isobutyl acetate, and
c) isolating crystalline Form IV of isobutyl acetate solvate of Compound-A.

Within the context of this embodiment, Compound-A is dissolved in suitable solvent at 65-80° C., in some particularly useful embodiments, at 70-75° C. Preferably, the suitable second solvent is a polar solvent. Examples of polar solvents include, but are not limited to, formic acid, acetic acid, methanol, ethanol, isopropanol, 1-propanol, n-butanol, 2-butanol, isobutanol, t-butanol, 2-methoxy ethanol, 2-ethoxy ethanol, dimethyl sulfoxide, N,N dimethyl formamide, N,N dimethyl acetamide, N-Methyl-2-pyrrolidone and mixtures thereof. In some particularly useful embodiments, formic acid is used as a solvent.

Isobutyl acetate solvent is added to the above reaction mixture. In some embodiments, it is found useful to maintain the reaction for about 2 hours to about 5 days. In some particularly useful embodiments the reaction is maintained for 1-3 days and in certain embodiments of the present disclosure, it is useful to maintain the reaction for 3 days.

Further, the solvent may be removed from the solution. Solvent removal may be carried out by techniques well known in the art, such as evaporation, distillation, vacuum drying or combinations thereof. In certain embodiments of the present disclosure, slow evaporation is useful for removing the solvent to isolate solid Form IV of isobutyl acetate solvate of Compound-A.

Next, Form IV of isobutyl acetate solvate of Compound-A may be isolated. This may be carried out by methods well-known in the art. For example, the suspension may be filtered to isolate solid Form IV of isobutyl acetate solvate of Compound-A.

In another embodiment, crystalline Form IV of isobutyl acetate solvate of Compound-A can be prepared by a process that includes the following steps:
a) suspending Compound-A in isobutyl acetate, and
b) isolating crystalline Form IV of isobutyl acetate solvate of Compound-A.

Within the context of this embodiment, Compound-A is suspended in isobutyl acetate solvent. In some embodiments, it is useful to maintain the reaction for about 2 hours to about 5 days. In some particularly useful embodiments the reaction is maintained for 1-3 days and, in certain embodiments of the present disclosure, it is useful to maintain the reaction for 16 to 24 hours.

Next, the solvent is removed from the suspension of Compound-A. Solvent removal may be carried out by techniques well known in the art, such as evaporation, distillation, vacuum drying or combinations thereof. In certain embodiments of the present disclosure vacuum drying of solvent is useful for removing the solvent to isolate crystalline Form IV of isobutyl acetate solvate of Compound-A.

In some embodiment of the present disclosure, the solid obtained after removing the solvent may be filtered and further subjected to drying to yield crystalline Form IV of isobutyl acetate solvate of Compound-A.

In one aspect, the present invention provides crystalline Form V of n-butyl acetate solvate of Compound-A.

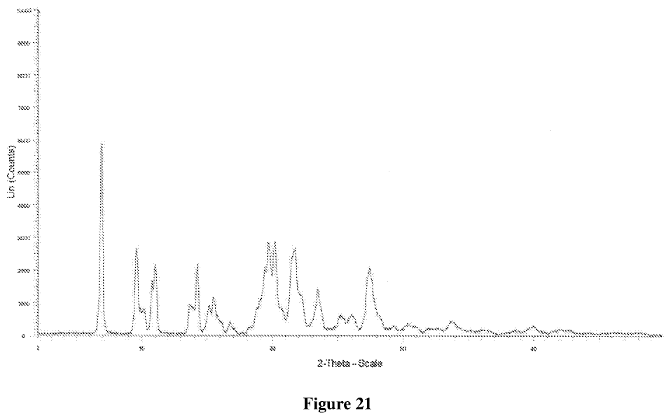

Within the context of the present invention, crystalline Form V of n-butyl acetate solvate of Compound-A prepared by methods disclosed herein may be characterized by PXRD spectrum depicted in FIG. 21 and having peaks (±0.2° 2θ) 6.82, 9.52, 10.75, 10.97 14.19, 19.39, 19.66, 20.12, 21.67, and 27.41.

The crystalline Form V of n-butyl acetate solvate of Compound-A prepared by methods disclosed herein may be characterized by the PXRD pattern in FIG. 21.

In another embodiment, crystalline Form V of n-butyl acetate solvate of Compound-A can be prepared by a process that includes the following steps:
a) dissolving Compound-A in suitable solvent,
b) adding n-butyl acetate, and
c) isolating crystalline Form V of n-butyl acetate solvate of Compound-A.

Within the context of this embodiment, Compound-A is dissolved in a suitable solvent at 65-80° C., in some particularly useful embodiments, at 70-75° C. Preferably, the suitable second solvent is a polar solvent. Examples of polar solvents include, but are not limited to, formic acid, acetic acid, methanol, ethanol, isopropanol, 1-propanol, n-butanol, 2-butanol, isobutanol, t-butanol, 2-methoxy ethanol, 2-ethoxy ethanol, dimethyl sulfoxide, N,N dimethyl formamide, N,N dimethyl acetamide, N-Methyl-2-pyrrolidone and mixtures thereof. In some particularly useful embodiments, formic acid is used as a solvent.

N-butyl acetate solvent is added to above reaction mixture and the solvent may be removed from the solution to isolate Form V of n-butyl acetate solvate of Compound-A. Solvent removal may be carried out by techniques well known in the art, such as evaporation, distillation, vacuum drying or combinations thereof. In certain embodiments of the present disclosure, slow evaporation is useful for removing the solvent to isolate crystalline Form V of n-butyl acetate solvate of Compound-A.

In some embodiments of the present disclosure, the solid obtained after removing the solvent may be filtered and further subjected to drying to yield crystalline Form V of n-butyl acetate solvate of Compound-A.

In another embodiment, crystalline Form V of n-butyl acetate solvate of Compound-A can be prepared by a process that includes the following steps:
a) suspending Compound-A in n-butyl acetate, and
b) isolating Form V of n-butyl acetate solvate of Compound-A.

Within the context of this embodiment, Compound-A is suspended in n-butyl acetate solvent. In some embodiments, it is found useful to maintain the reaction for about 2 hours to about 5 days, in some particularly useful embodiments, about 1-3 days and, in certain embodiments of the present disclosure, it is useful to maintain for 16 to 24 hours.

Next, the solvent is removed to isolate crystalline Form V of n-butyl acetate solvate of Compound-A. Solvent removal may be carried out by techniques well known in the art, such as evaporation, distillation, vacuum drying or combinations thereof. In certain embodiments of the present disclosure vacuum drying of solvent is useful for removing the solvent. In some embodiments of the present disclosure, the resulting solid is filtered to isolate Form V of n-butyl acetate solvate of Compound-A.

In one aspect, the present invention provides crystalline Form VI of toluene solvate of Compound-A.

Within the context of the present invention, crystalline Form VI of toluene solvate of Compound-A prepared by methods disclosed herein may be characterized by PXRD spectrum and having peaks (±0.2° 2θ) 6.66, 9.25, 10.01, 10.43, 10.85, 13.71, 13.96, 14.82, 15.99, 18.54, 19.10, 19.48, 20.05, 20.56, 21.36, 22.25, 23.54, 26.02, 27.33, and 28.04.

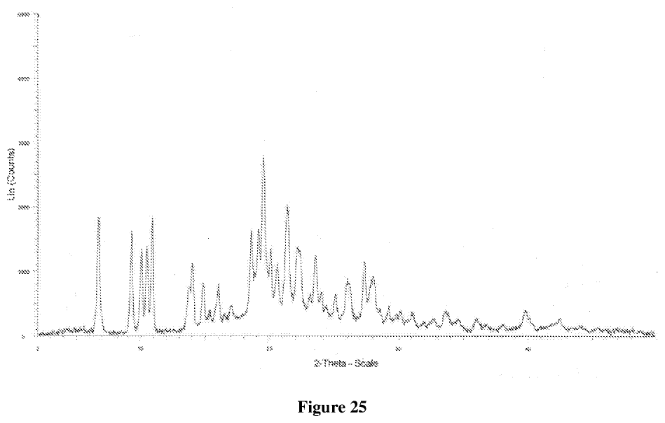

The crystalline Form VI of toluene solvate of Compound-A prepared by methods disclosed herein may be characterized by the PXRD pattern in FIG. 25.

In another embodiment, crystalline Form VI of toluene solvate of Compound-A can be prepared by a process that includes the following steps:
a) suspending Compound-A in toluene, and
b) isolating crystalline Form VI of toluene solvate of Compound-A.

Within the context of this embodiment, Compound-A is suspended in toluene. In some embodiments, it is found useful to maintain the reaction for about 2 hours to about 5 days, in some particularly useful embodiments about 1-3 days and in certain embodiments of the present disclosure, it is useful to maintain the reaction for 16 to 24 hours.

Next, the solvent is removed to isolate crystalline Form VI of toluene solvate of Compound-A. Solvent removal may be carried out by techniques well known in the art, such as evaporation, distillation, vacuum drying or combinations thereof. In certain embodiments of the present disclosure vacuum drying of solvent is useful for removing the solvent to isolate crystalline Form VI of toluene solvate of Compound-A.

In some embodiment of the present disclosure, the solid obtained after removing the solvent may be filtered and further subjected to drying to yield crystalline Form VI of toluene solvate of Compound-A.

In one aspect, the present invention provides crystalline Form VII of 4-methyl 2-pentanol solvate of Compound-A.

Within the context of the present invention, crystalline Form VII of 4-methyl 2-pentanol solvate of Compound-A prepared by methods disclosed herein may be characterized by PXRD spectrum and having peaks (±0. ° 2θ) 5.21, 6.20, 7.69, 12.50, 13.90, 14.09, 17.33, 19.36, 21.13, 21.71, and 23.04.

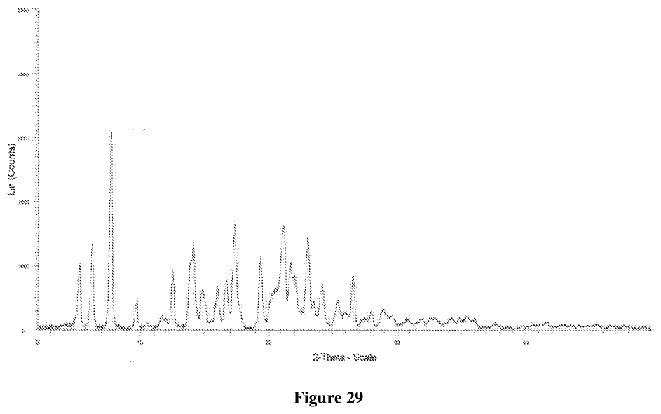

The crystalline Form VII of 4-methyl 2-pentanol solvate of Compound-A prepared by methods disclosed herein may be characterized by the PXRD pattern in FIG. 29.

In another embodiment, crystalline Form VII of 4-methyl 2-pentanol solvate of Compound-A can be prepared by a process that includes the following steps:
a) suspending Compound-A in 4-methyl 2-pentanol, and
b) isolating crystalline Form VII of 4-methyl 2-pentanol solvate of Compound-A.

Within the context of this embodiment, Compound-A is suspended in 4-methyl 2-pentanol solvent. In some embodiments, it is found useful to maintain the reaction for about 2 hours to about 5 days, in some particularly useful embodiments, about 1-3 days and, in certain embodiments of the present disclosure, it is useful to maintain for 16 to 24 hours.

Next, the solvent is removed from the suspension of Compound-A. Solvent removal may be carried out by techniques well known in the art, such as evaporation, distillation, vacuum drying or combinations thereof. In certain embodiments of the present disclosure vacuum drying of solvent is useful for removing the solvent to isolate crystalline Form VII of 4-methyl 2-pentanol solvate of Compound-A.

In some embodiments of the present disclosure, the solid obtained after removing the solvent may be filtered and further subjected to drying to yield crystalline Form VII of 4-methyl 2-pentanol solvate of Compound-A.

In one aspect, the present invention provides crystalline Form VIII of Compound-A.

Within the context of the present invention, crystalline Form VIII of Compound-A prepared by methods disclosed herein may be characterized by PXRD spectrum and having peaks (±0.2° 2θ) 7.51, 19.74, and 21.80.

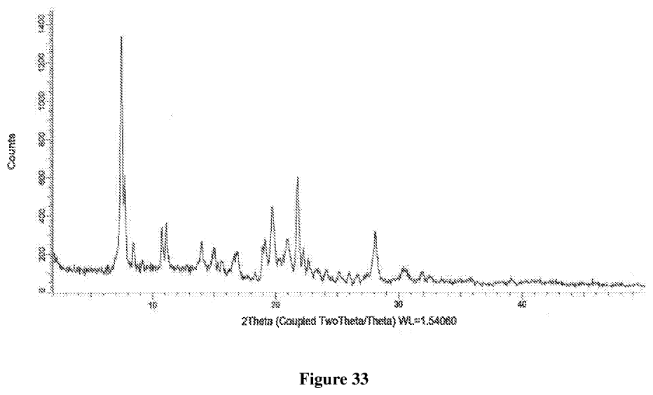

The crystalline Form VIII of Compound-A prepared by methods disclosed herein may be characterized by the PXRD pattern in FIG. 33.

In another embodiment, crystalline Form VIII of Compound-A can be prepared by a process that includes the following steps:
a) drying Form I of Compound-A, and
b) isolating crystalline Form VIII of Compound-A.

Within the context of the embodiment, Form I of Compound-a is subjected to drying by using techniques well known in the art, such as evaporation, distillation, vacuum drying or combinations thereof. In certain embodiments of the present disclosure vacuum drying is useful to isolate crystalline Form VIII of Compound-A.

In one aspect, the present invention provides crystalline Form IX of Compound-A.

Within the context of the present invention, crystalline Form IX of Compound-A prepared by methods disclosed herein may be characterized by PXRD spectrum having peaks (±0.2° 2θ) 6.73, 7.55, 21.00, 21.75, and 26.86.

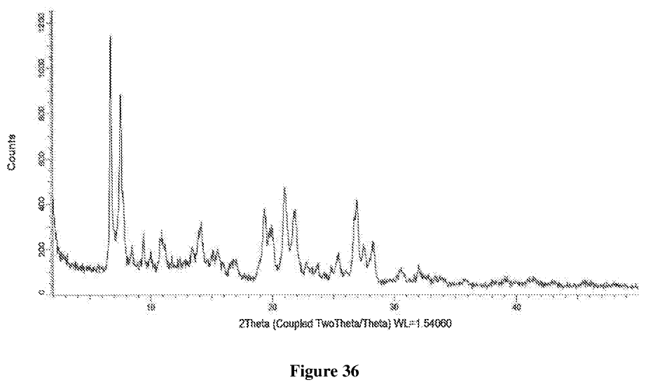

The crystalline Form IX of Compound-A prepared by methods disclosed herein may be characterized by the PXRD pattern in FIG. 36.

In another embodiment, crystalline Form IX of Compound-A can be prepared by a process that includes the following steps:
a) drying Form II of Compound-A, and
b) isolating crystalline Form IX of Compound-A.
   within the context of the embodiment, Form II of Compound-A is subjected to drying by using techniques well known in the art, such as evaporation, distillation, vacuum drying or combinations thereof. In certain embodiments of the present disclosure vacuum drying is useful to isolate crystalline Form IX of Compound-A.

In one aspect, the present invention provides crystalline Form X of n-propyl acetate solvate of Compound-A.

Within the context of the present invention, crystalline Form X of n-propyl acetate solvate of Compound-A prepared by methods disclosed herein may be characterized by PXRD spectrum having peaks (±0.2° 2θ) 6.73, 9.53, 10.87, 14.20, 15.46, 19.27, 19.50, 19.95, 21.41, 21.77, 23.46, 27.27, and 27.49.

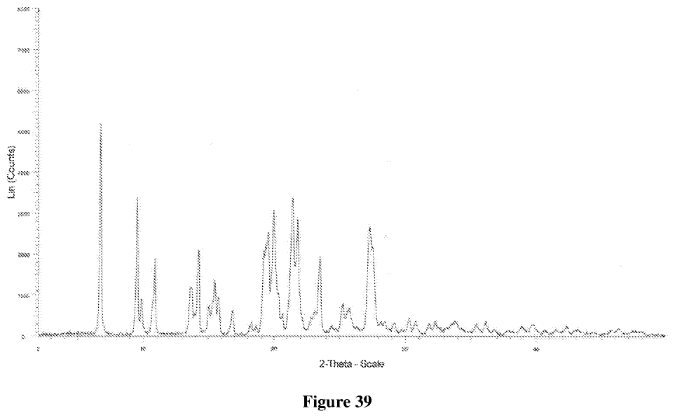

The crystalline Form X of n-propyl acetate solvate of Compound-A prepared by methods disclosed herein may be characterized by the PXRD pattern in FIG. 39.

In another embodiment, crystalline Form X of n-propyl acetate solvate of Compound-A can be prepared by a process that includes the following steps:
a) suspending Compound-A in n-propyl acetate, and
b) isolating crystalline Form X of n-propyl acetate solvate of Compound-A.

Within the context of this embodiment, Compound-A is suspended in n-propyl acetate. In some embodiments, it is found useful to maintain the reaction for about 2 hours to about 5 days, in some particularly useful embodiments, about 1-3 days and, in certain embodiments of the present disclosure, it is useful to maintain for 16 to 24 hours.

Next, the solvent is removed from the suspension of Compound-A. Solvent removal may be carried out by techniques well known in the art, such as evaporation, vacuum drying or combinations thereof. In certain embodiments of the present disclosure vacuum drying of solvent is useful for removing the solvent to isolate crystalline Form X of n-propyl acetate solvate of Compound-A.

In some embodiment of the present disclosure, the solid obtained after removing the solvent may be filtered and further subjected to drying to yield crystalline Form X of n-propyl acetate solvate of Compound-A.

In one aspect, the present invention provides crystalline Form XI of xylene solvate of Compound-A.

Within the context of the present invention, crystalline Form XI of xylene solvate of Compound-A prepared by methods disclosed herein may be characterized by PXRD spectrum having peaks (±0.2° 2θ) 6.84, 9.47, 10.20, 10.69, 11.05, 14.16, 19.24, 19.71, 21.49, 22.37, and 23.72.

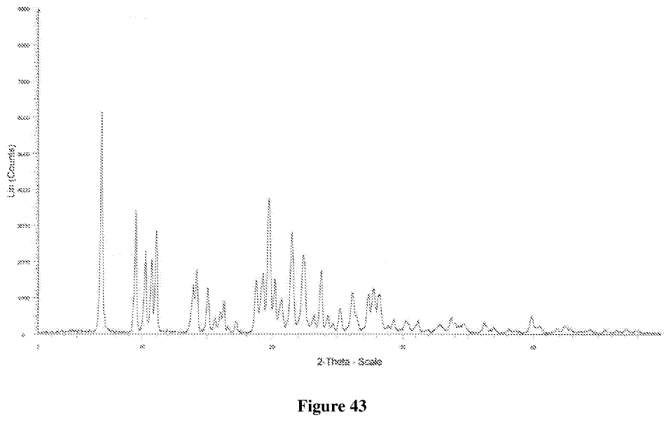

The crystalline Form XI of xylene solvate of Compound-A prepared by methods disclosed herein may be characterized by the PXRD pattern in FIG. 43.

In another embodiment, crystalline Form XI of xylene solvate of Compound-A can be prepared by a process that includes the following steps:
a) suspending Compound-A in xylene, and
b) isolating crystalline Form XI of xylene solvate of Compound-A.

Within the context of this embodiment, Compound-A is suspended in xylene. In some embodiments, it is found useful to maintain the reaction for about 2 hours to about 5 days, in some particularly useful embodiments, about 1-3 days and, in certain embodiments of the present disclosure, it is useful to maintain for 16 to 24 hours.

Next, the solvent is removed from the suspension of Compound-A. Solvent removal may be carried out by techniques well known in the art, such as evaporation, distillation, vacuum drying or combinations thereof. In certain embodiments of the present disclosure vacuum drying of solvent is useful for removing the solvent to isolate crystalline Form XI of xylene solvate of Compound-A.

In some embodiment of the present disclosure, the solid obtained after removing the solvent may be filtered and further subjected to drying to yield crystalline Form XI of xylene solvate of Compound-A.

In some useful embodiments of the present invention, the Compound-A used to prepare the solid form may be any form including, for example, amorphous form, crystalline form or solvate form.

### EXAMPLES

### Example 1: Processes for the Preparation of Amorphous Form of Compound-A

Compound-A (5 g) was dissolved in methanol (150 ml) at 60-65° C. The solution was filtered at 60-65° C. to remove undissolved particulate and then cooled to 25-30° C. The clear solution of Compound-A was subjected to spray drying in a laboratory Spray Dryer (Model Buchi-290) with a 5 ml/min feed rate of the solution and inlet temperature at 75° C. with 100% aspiration to yield an amorphous form of Compound-A.

### Example 2: Processes for the Preparation of Amorphous Form of Compound-A

Compound-A (0.5 g) was dissolved in methanol (10 ml) at 60-65° C. The clear solution was distilled to remove the solvent under vacuum completely using laboratory rotary evaporator at 40° C. to yield an amorphous form of Compound-A.

### Example 3: Processes for the Preparation of Crystalline Form I (Ethyl Acetate Solvate) of Compound-A

Compound-A (50 mg) was suspended in ethyl acetate (4 ml) and heated to 60° C. Undissolved material was then added formic acid (0.2 ml) at 60° C. to obtain a clear solution. The reaction mixture was cooled to 25-30° C. which allowed for slow solvent evaporation for 2 days without agitation. The solid obtained was filtered and dried under vacuum and identified as crystalline Form I (mono ethyl acetate solvate) of Compound-A.

### Example 4: Processes for the Preparation of Crystalline Form I (Ethyl Acetate Solvate) of Compound-A

Compound-A (3 g) was dissolved in formic acid (9.5 ml) at 70-75° C. The clear solution was cooled to 25-30° C. and then ethyl acetate (48 ml) was slowly added for 15-30 min. The reaction mixture was seeded with Form I (1%) and stirred for 3 days at 25-30° C. The resulting solid was filtered, washed with ethyl acetate (2 ml) and dried under vacuum at 30° C. for 15-30 min to obtain crystalline Form 1 (mono ethyl acetate solvate) of Compound-A.

Yield: 2.1 grams.

### Example 5: Processes for the Preparation of Crystalline Form II (Methyl n-Butyl Ketone Solvate) of Compound-A

An amorphous form of Compound-A (100 mg) was suspended in methyl n-butyl ketone (5 ml) and heated to 70-75° C.; further, maintained the same for 15 min under agitation. Material was not dissolved and then cooled the reaction mass to 20-25° C. and kept at the same temperature for 2 days without agitation. The solid obtained was filtered and identified as crystalline Form II (mono methyl n-butyl ketone solvate) of Compound-A.

### Example 6: Processes for the Preparation of Crystalline Form II (Methyl n-Butyl Ketone Solvate) of Compound-A

An amorphous form of Compound-A (100 mg) was suspended in methyl n-butyl ketone (5 ml) at 20-25° C. and agitated for 15 min. Material was not dissolved. The reaction mass was allowed to stand at the same temperature for 2 days without agitation. The solid obtained was filtered and identified as crystalline Form II (mono methyl n-butyl ketone solvate) of Compound-A.

### Example 7: Processes for the Preparation of Crystalline Form II (Methyl n-Butyl Ketone Solvate) of Compound-A

An amorphous form of Compound-A (100 mg) was suspended in methyl n-butyl ketone (5 ml) at 20-25° C. Material was not dissolved. The reaction mass was allowed to stand at −20° C. for 2 days without agitation. The solid obtained was filtered and identified as crystalline Form II (mono methyl n-butyl ketone solvate) of Compound-A.

### Example 8: Processes for the Preparation of Crystalline Form II (Methyl n-Butyl Ketone Solvate) of Compound-A

An amorphous form of Compound-A (100 mg) was suspended in methyl n-butyl ketone (5 ml) at 20-25° C. The reaction mass was heated to 70-75° C. Undissolved material was then added methanol (0.5 ml) at 70-75° C. to obtain a clear solution. The clear solution was allowed to stand at 25-30° C. for 2 days without agitation. The solid obtained was filtered and identified as crystalline Form II (mono methyl n-butyl ketone solvate) of Compound-A.

### Example 9: Processes for the Preparation of Crystalline Form II (Methyl n-Butyl Ketone Solvate) of Compound-A

An amorphous form of Compound-A (3 g) was suspended in methyl n-butyl ketone (30 ml) at 25-30° C. and stirred at the same temperature for 3 days. The solid obtained was filtered, washed with methyl n-butyl ketone (2 ml) and dried under vacuum at 30° C. for 15-30 min to obtain crystalline Form II (mono methyl n-butyl ketone solvate) of Compound-A.

### Example 10: Processes for the Preparation of Crystalline Form III (Anisole Solvate) of Compound-A

Compound-A (0.1 g) was dissolved in formic acid (0.3 ml) at 75° C. resulting in a clear solution to which anisole (5 ml) was added. The solution was allowed to evaporate slowly for 3 days at 20-25° C. without agitation. The solid obtained was filtered and identified as crystalline Form III of Compound-A.

### Example 11: Processes for the Preparation of Crystalline Form III (Anisole Solvate) of Compound-A

An amorphous form of Compound-A (50 mg) was suspended in anisole (0.4 ml) at 20-25° C. and the suspension was maintained while shaking at 20° C. for 24 hours. The reaction mass was then kept in a vacuum tray dryer and dried at 30° C. for 24 hours. The resulting solid was identified as crystalline Form III of Compound-A.

### Example 12: Processes for the Preparation of Crystalline Form III (Anisole Solvate) of Compound-A

An amorphous form of Compound-A (2.0 g) was suspended in anisole (20 ml) at 20-25° C. and stirred at the same temperature for 16 hours. The reaction mass was filtered and dried in a vacuum tray dryer at 30° C. for 2 hours. The resulting solid was identified as crystalline Form III of Compound-A.

### Example 13: Processes for the Preparation of Crystalline Form IV (Isobutyl Acetate Solvate) of Compound-A

Compound-A (0.1 g) was dissolved in formic acid (0.3 ml) at 75° C. To this clear solution isobutyl acetate (5 ml) was added. The solution was allowed to slowly evaporate at 20-25° C. for 3 days. The solid obtained was filtered and identified as crystalline Form IV of Compound-A.

### Example 14: Processes for the Preparation of Crystalline Form IV (Isobutyl Acetate Solvate) of Compound-A

An amorphous form of Compound-A (50 mg) was suspended in isobutyl acetate (0.4 ml) at 20-25° C. and, while shaking, the suspension was maintained at 20° C. for 24 hours. The reaction mass was then kept in a vacuum tray dryer at 30° C. for 24 hours. The resulting solid was identified as crystalline Form IV of Compound-A.

### Example 15: Processes for the Preparation of Crystalline Form IV (Isobutyl Acetate Solvate) of Compound-A

An amorphous form of Compound-A (2.0 g) was suspended in isobutyl acetate (20 ml) at 20-25° C. and stirred at the same temperature for 16 hours. The resulting solid was filtered and dried in a vacuum tray dryer at 30° C. for 2 hours and identified as crystalline Form IV of Compound-A.

### Example 16: Processes for the Preparation of Crystalline Form V (n-Butyl Acetate Solvate) of Compound-A

Compound-A (0.1 g) was dissolved in formic acid (0.3 ml) at 75° C. To this clear solution n-butyl acetate (5 ml) was added and the solution was allowed to evaporate at 20-25° C. for 3 days.

The solid obtained was filtered and identified as crystalline Form V of Compound-A.

### Example 17: Processes for the Preparation of Crystalline Form V (n-Butyl Acetate Solvate) of Compound-A

An amorphous form of Compound-A (2.0 g) was suspended in n-butyl acetate (20 ml) at 20-25° C. and stirred at the same temperature for 16 hours. The product was filtered and dried in a vacuum tray dryer at 30° C. for 2 hours. The resulting solid was identified as crystalline Form V of Compound-A.

### Example 18: Processes for the Preparation of Crystalline Form VI (Toluene Solvate) of Compound-A

An amorphous form of Compound-A (50 mg) was suspended in toluene (0.4 ml) at 20-25° C. and, while shaking, the suspension was maintained at 20° C. for 24 hours. The reaction mass was then kept in a vacuum tray dryer at 30° C. for 24 hours. The resulting solid was identified as crystalline Form VI of Compound-A.

### Example 19: Processes for the Preparation of Crystalline Form VI (Toluene Solvate) of Compound-A

An amorphous form of Compound-A (2.0 g) was suspended in toluene (20 ml) at 20-25° C. and stirred at the same temperature for 16 hours. The product was filtered and dried in a vacuum tray dryer at 30° C. for 2 hours and the resulting solid was identified as crystalline Form VI of Compound-A.

### Example 20: Processes for the Preparation of Crystalline Form VII (4-Methyl 2-Pentanol solvate) of Compound-A

An amorphous form of Compound-A (50 mg) was suspended in 4-methyl-2-pentanol (0.4 ml) at 20-25° C. and, while shaking, the suspension was maintained at 20° C. for 24 hours. The reaction mass was then kept in a vacuum tray dryer at 30° C. for 24 hours. The resulting solid was identified as crystalline Form VII of Compound-A.

### Example 21: Processes for the Preparation of Crystalline Form VII (4-Methyl 2-Pentanol Solvate) of Compound-A

An amorphous form of Compound-A (2.0 g) was suspended in 4-methyl-2-pentanol (20 ml) at 20-25° C. The suspension was then stirred at the same temperature for 16 hours. The product obtained was filtered and dried in a vacuum tray dryer at 30° C. for 2 hours. The resulting solid was identified as crystalline Form VII of Compound-A.

### Example 22: Processes for the Preparation of Crystalline Form VIII of Compound-A

Form I of Compound-A (200 mg) was placed in a petri-dish and dried in a vacuum tray dryer at 70-100° C. for 15 hours. The resulting solid was identified as crystalline Form VIII of Compound-A.

### Example 23: Processes for the Preparation of Crystalline Form IX of Compound-A

Form II of Compound-A (200 mg) was placed in a petri-dish and dried in a vacuum tray dryer at 40-70° C. for 15-24 hours. The resulting solid was identified as crystalline Form IX of Compound-A.

### Example 24: Processes for the Preparation of Crystalline Form X (n-Propyl Acetate Solvate) of Compound-A

An amorphous form of Compound-A (50 mg) was suspended in n-propyl acetate (0.4 ml) at 20-25° C. and, while shaking, the suspension was maintained at 20° C. for 24 hours. The reaction mass was then kept in a vacuum tray dryer at 30° C. for 24 hours. The resulting solid was identified as crystalline Form X of Compound-A.

### Example 25: Processes for the Preparation of Crystalline Form X (n-Propyl Acetate Solvate) of Compound-A

An amorphous form of Compound-A (2.0 g) was suspended in n-propyl acetate (20 ml) at 20-25° C. and the suspension was stirred at the same temperature for 16 hours. The product obtained was filtered and dried in a vacuum tray dryer at 30° C. for 2 hours. The resulting solid was identified as crystalline Form X of Compound-A.

### Example 26: Processes for the Preparation of Crystalline Form XI (Xylene Solvate) of Compound-A

An amorphous form of Compound-A (50 mg) was suspended in xylene (0.4 ml) at 20-25° C. and, while shaking, the suspension was maintained at 20° C. for 24 hours. The reaction mass was then kept in a vacuum tray dryer at 30° C. for 24 hours. The resulting solid was identified as crystalline Form XI of Compound-A.

### Example 27: Processes for the Preparation of Crystalline Form XI (Xylene Solvate) of Compound-A

An amorphous form of Compound-A (2.0 g) was suspended in xylene (20 ml) at 20-25° C. and the suspension was stirred at the same temperature for 16 hours. The product obtained was filtered and dried in a vacuum tray dryer at 30° C. for 2 hours. The resulting solid was identified as crystalline Form XI of Compound-A.

### Example 28: Process for the Preparation of Crystalline Form of Compound-A

Form II of Compound-A (200 mg) obtained as above examples was placed in a petri-dish and dried in a vacuum tray dryer at 100-110° C. for 1-2 days. The resulting solid was identified as the crystalline form of Compound-A disclosed in U.S. Pat. No. 8,263,583.

1-2. (canceled)

3. A process for the preparation of amorphous form of (5-[3-(3-Hydroxyphenoxy)azetidin-1-yl]-5-methyl-2,2-diphenylhexanamide hydrochloride comprising the steps of:
a) dissolving (5-[3-(3-Hydroxyphenoxy)azetidin-1-yl]-5-methyl-2,2-diphenylhexanamide hydrochloride in a solvent; and
b) removing the solvent to isolate amorphous form of (5-[3-(3-Hydroxyphenoxy)azetidin-1-yl]-5-methyl-2,2-diphenylhexanamide hydrochloride.

4. The process according to the claim 3, wherein the solvent is selected from the group consisting of methanol, ethanol, isopropanol, 1-propanol, n-butanol, 2-butanol, isobutanol, t-butanol, 2-methoxy ethanol, 2-ethoxy ethanol, and mixtures thereof.

5-42. (canceled)

43. A polymorphic form of (5-[3-(3-hydroxyphenoxy)azetidin-1-yl]-5-methyl-2,2-diphenylhexanamide hydrochloride selected from the group consisting of:
a) amorphous form;
b) crystalline Form I characterized by a PXRD pattern having peaks 6.83, 9.58, 10.91, 14.29, 19.71, 20.04, 21.59, 22.14, and 27.65±0.2° 2Θ;
c) crystalline Form II characterized by a PXRD pattern having peaks 6.61, 20.80, and 26.81±0.2° 2Θ;
d) crystalline Form III characterized by a PXRD pattern having peaks 6.86, 19.7 and 22.36±0.2° 2Θ;
e) crystalline Form IV characterized by a PXRD pattern having peaks 6.68, 8.92, 11.01, 13.32, 13.83, 15.80, 18.60, 19.26, 19.56, 20.11, 20.37, 22.10, 23.76, 25.16, 25.96, 26.94, and 28.00±0.2° 2Θ;
f) crystalline Form V characterized by a PXRD pattern having peaks 6.82, 9.52, 10.75, 10.97 14.19, 19.39, 19.66, 20.12, 21.67, and 27.41±0.2° 2Θ;
g) crystalline Form VI characterized by a PXRD pattern having peaks 6.66, 9.25, 10.01, 10.43, 10.85, 13.71, 13.96, 14.82, 15.99, 18.54, 19.10, 19.48, 20.05, 20.56, 21.36, 22.25, 23.54, 26.02, 27.33, and 28.04±0.2° 2Θ;
h) crystalline Form VII characterized by a PXRD pattern having peaks 5.21, 6.20, 7.69, 12.50, 13.90, 14.09, 17.33, 19.36, 21.13, 21.71, and 23.04±0.2° 2Θ;
i) crystalline Form VIII characterized by a PXRD pattern having peaks 7.51, 19.74, and 21.80±0.2° 2Θ;
j) crystalline Form IX characterized by a PXRD pattern having peaks 6.73, 7.55, 21.00, 21.75, and 26.86±0.2° 2Θ;
k) crystalline Form X characterized by a PXRD pattern having peaks 6.73, 9.53, 10.87, 14.0, 15.46, 19.27, 19.50, 19.95, 21.41, 21.77, 23.46, 27.27, and 27.49±0.2° 2Θ; and
l) crystalline Form XI characterized by a PXRD pattern having peaks 6.84, 9.47, 10.20, 10.69, 11.05, 14.16, 19.24, 19.71, 21.49, 22.37, and 23.72±0.2° 2Θ.

44. The polymorphic form of (5-[3-(3-hydroxyphenoxy)azetidin-1-yl]-5-methyl-2,2-diphenylhexanamide hydrochloride of claim 43, wherein
a) the amorphous form is characterized by a PXRD pattern as shown in FIG. 1;
b) Form I is characterized by a PXRD pattern as shown in FIG. 5;
c) Form II is characterized by a PXRD pattern as shown in FIG. 9;
d) Form III is characterized by a PXRD pattern as shown in FIG. 13;
e) Form IV is characterized by a PXRD pattern as shown in FIG. 17;
f) Form V is characterized by a PXRD pattern as shown in FIG. 21;
g) Form VI is characterized by a PXRD pattern as shown in FIG. 25;
h) Form VII is characterized by a PXRD pattern as shown in FIG. 29;
i) Form VIII is characterized by a PXRD pattern as shown in FIG. 33;
j) Form IX is characterized by a PXRD pattern as shown in FIG. 36;
k) Form X is characterized by a PXRD pattern as shown in FIG. 39; and
l) Form XI is characterized by a PXRD pattern as shown in FIG. 43.

45. A process for preparing a crystalline form of 5-[3-(3-Hydroxyphenoxy)azetidin-1-yl]-5-methyl-2,2-diphenylhexanamide hydrochloride, comprising
a) suspending (5-[3-(3-Hydroxyphenoxy)azetidin-1-yl]-5-methyl-2,2-diphenyl hexanamide hydrochloride a solvent, and
b) isolating a crystalline form of (5-[3-(3-Hydroxyphenoxy)azetidin-1-yl]-5-methyl-2,2-diphenyl hexanamide hydrochloride,
wherein
when the solvent is methyl n-butyl ketone, the crystalline form is Form II;
when the solvent is anisole, the crystalline form is Form III;
when the solvent is isobutyl acetate, the crystalline form is Form IV;
when the solvent is n-butyl acetate, the crystalline form is Form V;
when the solvent is toluene, the crystalline form is Form VI;
when the solvent is 4-methyl 2-pentanol, the crystalline form is Form VII;
when the solvent is n-propyl acetate, the crystalline form is Form X; and
when the solvent is xylene, the crystalline form is Form XI.

46. A process for preparing a crystalline form of 5-[3-(3-hydroxyphenoxy)azetidin-1-yl]-5-methyl-2,2-diphenylhexanamide hydrochloride, comprising
a) suspending (5-[3-(3-hydroxyphenoxy)azetidin-1-yl]-5-methyl-2,2-diphenylhexanamide hydrochloride in a first solvent,
b) adding a second solvent,
c) optionally cooling the reaction mixture; and
c) isolating the crystalline form of (5-[3-(3-hydroxyphenoxy)azetidin-1-yl]-5-methyl-2,2-diphenylhexanamide hydrochloride,
wherein
when the first solvent is ethyl acetate, the crystalline form is Form I and the reaction mixture is cooled; and
when the first solvent is methyl n-butyl ketone, the crystalline form is Form II.

47. A process for preparing a crystalline form of 5-[3-(3-hydroxyphenoxy)azetidin-1-yl]-5-methyl-2,2-diphenylhexanamide hydrochloride, comprising
a) drying a first crystalline form of (5-[3-(3-hydroxyphenoxy)azetidin-1-yl]-5-methyl-2,2-diphenylhexanamide hydrochloride, and
b) isolating a second crystalline form of (5-[3-(3-hydroxyphenoxy)azetidin-1-yl]-5-methyl-2,2-diphenylhexanamide hydrochloride,
wherein
when the first crystalline form is Form I, the second crystalline form is Form VIII; and
when the first crystalline form is Form II, the second crystalline form is Form IX.

48. A process for preparing a crystalline form of 5-[3-(3-hydroxyphenoxy)azetidin-1-yl]-5-methyl-2,2-diphenylhexanamide hydrochloride, comprising
a) dissolving (5-[3-(3-hydroxyphenoxy)azetidin-1-yl]-5-methyl-2,2-diphenylhexanamide hydrochloride in a first solvent,
b) adding a second solvent, and
c) isolating the crystalline form of (5-[3-(3-hydroxyphenoxy)azetidin-1-yl]-5-methyl-2,2-diphenylhexanamide hydrochloride,
wherein
when the second solvent is ethyl acetate, the crystalline form is Form I;
when the second solvent is anisole, the crystalline form is Form III;
when the second solvent is isobutyl acetate, the crystalline form is Form IV; and
when the second solvent is n-butyl acetate, the crystalline form is Form V.
